(19) United States
(12) Patent Application Publication
(10) Pub. No.: US 2025/0072861 A1
(43) Pub. Date: Mar. 6, 2025

(54) SYSTEM FOR LOCALIZING AND MONITORING BIOPSY AND ABLATION NEEDLES WITHIN THE BODY

(71) Applicant: California Institute of Technology, Pasadena, CA (US)

(72) Inventors: David C. Garrett, Glendale, CA (US); Jinhua Xu, Pasadena, CA (US); Geng Ku, South Pasadena, CA (US); Lihong Wang, Arcadia, CA (US)

(21) Appl. No.: 18/825,733

(22) Filed: Sep. 5, 2024

Related U.S. Application Data
(60) Provisional application No. 63/536,661, filed on Sep. 5, 2023.

(51) Int. Cl. A61B 8/08

(52) U.S. Cl. CPC A61B 8/0841

(57) ABSTRACT

Imaging methods and apparatus for localizing biopsy needles coupled to an ultrasonic transmitter and/or monitoring ablation of an ablation probe connected to a microwave source, which include an arbitrary waveform generator for generating a chirp signal that excites the ultrasonic transmitter to generate an ultrasonic signal or modulates the microwave signal.

### CROSS-REFERENCES TO RELATED APPLICATION

This application claims priority to and benefit of U.S. Provisional Patent Application No. 63/536,661, titled “System for Localizing and Monitoring Biopsy and Ablation Needles within the Body,” filed on Sep. 5, 2023, which is hereby incorporated by reference in its entirety and for all purposes.

### FEDERALLY SPONSORED RESEARCH OR DEVELOPMENT

This invention was made with government support under Grant No. CA220436awarded by the National Institutes of Health. The government has certain rights in the invention.

### FIELD

Certain aspects generally relate to the field of medical imaging, diagnosis, and/or treatment. More specifically, some aspects relate to localizing biopsy needles using ultrasound imaging and/or tracking the progress of needle microwave/radiofrequency/optical ablation using thermoacoustic imaging.

### BACKGROUND

Needle biopsy can be used to extract a small amount of tissue or fluid from regions suspected of being cancerous or suffering from other conditions. To guide positioning of the biopsy needle with respect to internal features, ultrasound and x-ray computed tomography (CT) have been used. However, current methods of ultrasound localization only function properly for relatively superficial targets since these ultrasound imaging methods use conventional ultrasonic probes and operate based on passive scattering from the biopsy needle. Guiding a biopsy needle using CT imaging enables whole-body localization, but requires iterative positioning and can lead to harmful radiation to the patient.

Microwave ablation is a clinical technique used to thermally treat one or more cancerous regions to kill growing tumor cells. A needle may be positioned in a targeted area, then microwave signals are coupled through the needle to heat a cm-scale region near the tip. Currently, ablation procedures use prescribed durations and microwave power. However, subject- and region-specific tissue differences can result in different heating rates and non-uniform ablation.

### SUMMARY

Certain aspects relate to methods and apparatus for medical imaging, diagnosis, and/or treatment. More specifically, some aspects relate to methods and apparatus for localizing biopsy needles using ultrasound imaging and/or tracking the progress of needle ablation using thermoacoustic imaging. Various forms of needle ablation may be used including microwave or other radiofrequency ablation, optical ablation, ultrasonic ablation, etc.

In some embodiments, acoustic signals are coupled into a biopsy needle and signals are detected outside the body to determine the location of the needle tip. The needle tip location may be superimposed onto a structural image obtained with whole-body ultrasound imaging such as may be acquired using ultrasound tomography (UST) techniques.

In some embodiments, a microwave ablation probe (also sometimes referred herein as a needle) is excited using modulated microwave signals. Transient heating in the tissue generates thermoacoustic signals, which are used to generate thermoacoustic images of the local heating profile around the ablation needle. In other embodiments, the radiation can be extended to other forms such as other radiofrequency, optical, and ultrasonic waves.

Certain aspects are directed to imaging apparatus having one or more energy sources configured to generate energy waves and an arbitrary waveform generator configured to generate one or more chirp signals. The arbitrary waveform generator is connected to the one or more energy sources. The one or more chirp signals modulate the energy waves to generate modulated energy waves. The imaging apparatus also includes a needle comprising a needle tip. The needle is connected to a first energy source of the energy sources. The is configured to emit the modulated energy waves at or near the needle tip. The imaging apparatus also includes an ultrasonic detector array configured to detect ultrasound signals or thermoacoustic signals based on the modulated energy waves emitted. In some cases, the needle is a biopsy needle, the first energy source includes a first ultrasonic transmitter acoustically coupled to the biopsy needle, and the ultrasonic detector array is configured to detect a first set of ultrasound signals while the biopsy needle emits acoustic signals at the needle tip. In some other cases, the first energy source is a microwave source and the arbitrary waveform generator and the microwave source are configured to provide a modulated microwave signal. In these other cases, the needle may be an ablation probe configured to emit the modulated microwave signal at a radiating portion of the ablation probe and the ultrasonic detector array may be configured to detect thermoacoustic signals from thermoelastic expansion based on the modulated microwave signal emitted from the radiating portion.

Certain aspects are directed to an imaging method that includes causing an ultrasonic transmitter coupled to a biopsy needle to emit acoustic waves from a needle tip inserted into a subject and determining a location of the needle tip within the subject using a first set of ultrasonic signals detected by an ultrasonic detector array while the biopsy needle emits the acoustic waves.

Certain aspects are directed to an imaging method that includes (a) causing modulation of energy waves from an energy source using one or more chirp signals from an arbitrary waveform generator to generate a modulated energy wave signal, the modulated energy wave signal propagated to a radiating portion of an ablation probe, (b) generating a plurality of two-dimensional thermoacoustic images from thermoacoustic signals detected by an ultrasonic detector array, and (c) determining an indicator of ablation progress from the two-dimensional thermoacoustic images. These and other features are described in more detail below with reference to the associated drawings.

### BRIEF DESCRIPTION OF THE DRAWINGS

Certain aspects are illustrated by way of example and not limitation in the figures of the accompanying drawings in which like references indicate similar elements.

FIG. 1 depicts a block diagram of an ultrasound tomography (UST) system, according to embodiments.

FIG. 2A depicts an example of a reflection-mode image of a human abdomen resulting from a full imaging operation of the UST system shown in FIG. 1, according to an embodiment.

FIG. 2B depicts an example of an image of a speed of sound profile overlaid on the reflectivity image from FIG. 2A, according to an embodiment.

FIG. 3A includes reflection-mode images of the legs of a human body acquired using the UST system in FIG. 1, according to an embodiment.

FIG. 3B includes speed of sound and attenuation coefficient profiles of a human abdomen acquired using the UST system in FIG. 1, according to an embodiment.

FIG. 4 depicts a block diagram of components of a biopsy needle localization system, according to various implementations.

FIG. 5 depicts a cross-sectional view of a biopsy needle localization device, according to embodiments.

FIG. 6 depicts an isometric view of the biopsy needle localization device in FIG. 5.

FIG. 7A depicts an isometric view of components of a biopsy needle localization system, according to embodiments.

FIG. 7B depicts a cross-sectional view of an acoustic receiver of an acoustic detector array, according to an embodiment.

FIG. 8A is an example image frame of the location of biopsy needle superimposed onto a UST tomographic image generated by a biopsy needle localization system, according to an embodiment.

FIG. 8B is a photograph of the experimental setup of the biopsy needle localization system, according to an embodiment.

FIG. 9 depicts a flowchart of a biopsy needle localization method, according to various embodiments.

FIG. 10 depicts a block diagram of components of a needle ablation monitoring system, according to various implementations.

FIG. 11 depicts a side view of a needle ablation monitoring device for use with thermoacoustic tomography (TAT) ablation monitoring, according to embodiments.

FIG. 12 depicts an isometric view of components of a needle ablation monitoring system, according to embodiments.

FIG. 13 depicts a block diagram of components of a needle ablation monitoring system, according to various implementations.

FIG. 14 depicts an example of a thermoacoustic image of bovine liver tissue acquired by needle ablation monitoring system in FIG. 13, according to an implementation.

FIG. 15 depicts a graph of maximum amplitudes of the thermoacoustic images taken every minute of heating time (minutes) over a duration of 5 minutes, according to an implementation.

FIG. 16 is a photograph of the bovine liver after needle ablation showing a treated region, according to an implementation.

FIG. 17 depicts a flowchart of a needle ablation monitoring method, according to various embodiments.

FIG. 18 depicts a block diagram of components of tomography imaging system for needle localization and/or needle ablation monitoring, according to various embodiments.

FIG. 19 depicts an example of components of a computing device, according to embodiments.

These and other features are described in more detail below with reference to the associated drawings.

### DETAILED DESCRIPTION

Different aspects are described below with reference to the accompanying drawings. The features illustrated in the drawings may not be to scale. In the following description, numerous specific details are set forth in order to provide a thorough understanding of the presented embodiments. The disclosed embodiments may be practiced without one or more of these specific details. In other instances, well-known operations have not been described in detail to avoid unnecessarily obscuring the disclosed embodiments. While the disclosed embodiments will be described in conjunction with the specific embodiments, it will be understood that it is not intended to limit the disclosed embodiments.

Various embodiments and aspects will be described with reference to details discussed below, and the accompanying drawings will illustrate the various embodiments. The following description and drawings are illustrative and are not to be construed as limiting. Numerous specific details are described to provide a thorough understanding of various embodiments of the present invention. However, in certain instances, well-known or conventional details are not described to provide a concise discussion of embodiments of the present inventions.

Reference in the specification to “one embodiment” or “an embodiment” or “another embodiment” means that a particular feature, structure, or characteristic described in conjunction with the embodiment can be included in at least one embodiment. The appearances of the phrase “in one embodiment” in various places in the specification do not necessarily all refer to the same embodiment.

### I. Ultrasound and Thermoacoustic Tomography

Certain embodiments pertain to ultrasound tomography (UST) or thermoacoustic tomography (TAT) systems and methods. These techniques can be used to acquire cross-sectional images of an entire body or other subject for “whole-body imaging” or cross-sectional images of portions of a subject. Although certain implementations are described herein as acquiring cross-sectional images of an entire subject, it would be understood that these implementations can also be used to acquire cross-sectional images of a portion. The UST systems enable whole body ultrasound tomography of subjects in both reflection and transmission modes. Both reflected signals and transmitted signals may be captured simultaneously. In certain embodiments, to generate 2D isotropically resolved images across the entire cross-section in vivo, a circular ultrasound detector array and a rotating ultrasonic transmitter are employed.

To help guide diagnosis and treatment of various conditions, certain implementations described herein involve techniques for localizing biopsy needles within a body (e.g., within the abdomen) and/or techniques for tracking the progress of needle ablation using thermoacoustic imaging. According to various aspects, needle ablation may be performed using microwave ablation or other radio frequency ablation, optical ablation such as laser ablation, ultrasound ablation, or other suitable ablation technique. Some examples of techniques for localizing biopsy needles are described in Section II. These techniques can be used to determine the location of the tip of the biopsy needle in vivo to guide the biopsy needle during a biopsy procedure. The location of the tip may be superimposed onto a cross-sectional image of the subject acquired using UST to guide the biopsy needle to the biopsy location, for example. Some examples of techniques for monitoring needle ablation using TAT are described in Section III. These techniques may be performed in vivo to track the progress of ablation at the needle tip. This suite of techniques may provide a convenient tool for screening and treating a variety of conditions.

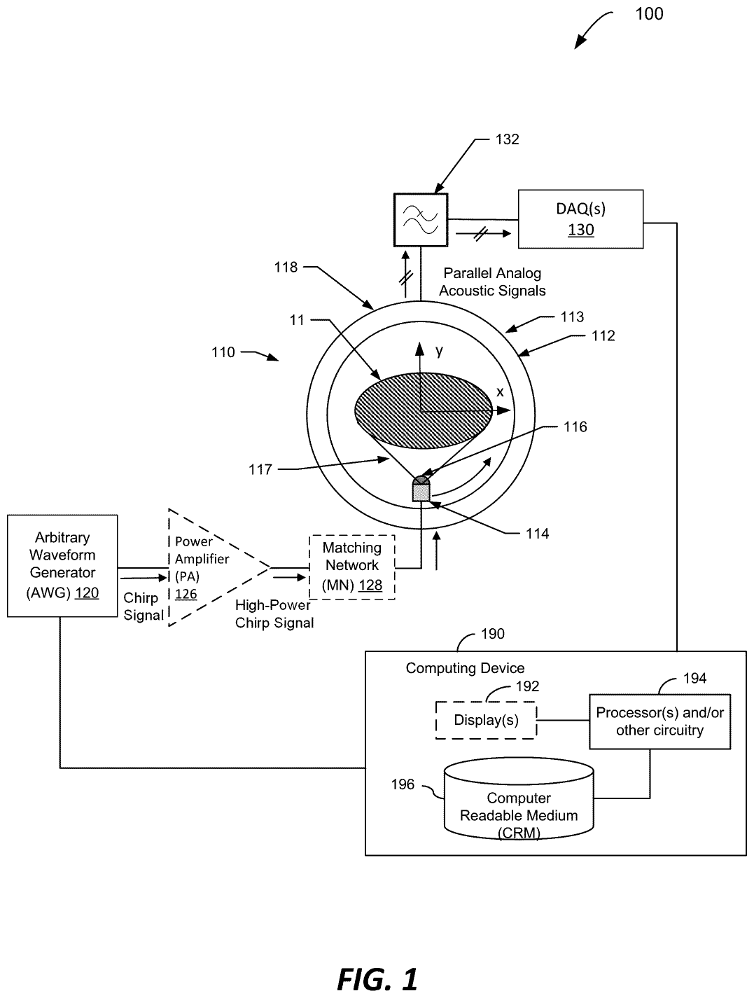

FIG. 1 depicts a block diagram of components of an ultrasound tomography (UST) system 100, according to various embodiments. UST system 100 includes an ultrasonic detector array assembly 110 with an ultrasonic detector array 112 that includes a plurality of ultrasonic receivers 113 (also sometimes referred to herein as “transducer elements”) that detect ultrasound signals. Ultrasonic detector array assembly 110 also includes an ultrasonic transmitter 114 (e.g., a single element transmitter/transducer) having a diverging lens 116 with a field of view 117. An example of a suitable ultrasonic transmitter is a single clement 2.25 MHz ultrasonic transducer element having a 1.5 inch diameter such as the V395 transducer made by Olympus. An example of a suitable diverging lens is a cylindrical diverging polymethylpentene (TPX) lens. The UST system 100 also includes an x-axis and a y-axis at a plane at ultrasonic receivers 113 of the ultrasonic detector array 112. The UST system 100 also includes a z-axis (not shown) perpendicular to the plane formed by the x-axis and the y-axis.

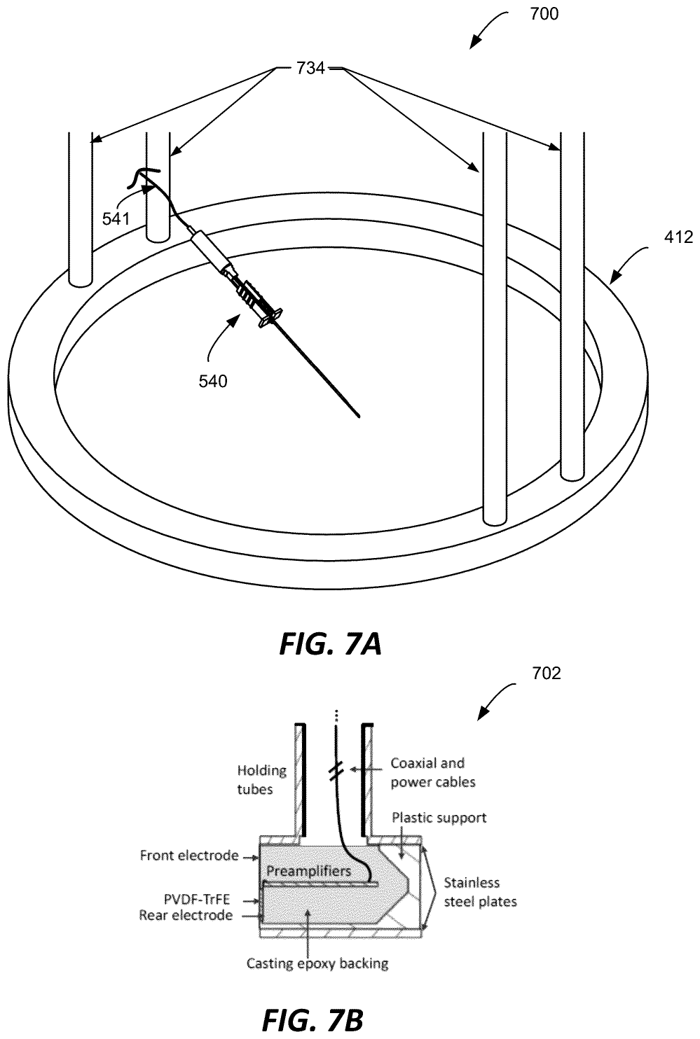

In the example shown in FIG. 1 and in other embodiments, the systems include an ultrasonic detector array with a plurality of ultrasonic receivers (transducers). In certain cases, the ultrasonic receivers are arranged in a circular (full-ring) array. An example of a suitable diameter of a circular array is about 60 cm. Other examples of suitable diameters are more than 60 cm. In other cases, other geometries may be used such as a linear array, a hemispherical array, an arc-shaped array, a two-dimensional rectangular array, or other geometrical arrangement. In some implementations, multiple arrays may be used such as a plurality of arc-shaped arrays forming a full circle (360 degree view). The ultrasonic detector array of these various embodiments may have any suitable number of ultrasonic receivers (e.g., 256, 512, 100, 200, 1024, etc.). In one aspect, an ultrasonic detector array has 512 ultrasonic receivers. The ultrasonic receivers may be distributed equally around the array geometry with a constant or nearly constant pitch between adjacent receivers. FIG. 7B depicts a cross-sectional view of an example of an acoustic receiver of an acoustic detector array.

In various embodiments, an ultrasonic detector array may include integrated pre-amplifiers coupled to the ultrasonic receivers or the pre-amplifiers may be separate and connected to the ultrasonic receivers. In one example, an acoustic detector array includes a plastic disc (e.g., a 60 cm diameter machined plastic disc) that is used as a mold for the inner surface of the array. The ultrasonic receiver elements and preamplifiers may be housed in a stainless-steel shielded enclosure, with coaxial cables for each element connected through stainless steel holding tubes. Casting epoxy may be used as a backing material for each element, and an angled back panel is used to reduce reverberation. In an example with integrated digitizers, the integrated digitizers may be controlled and transfer data through USB over optical fiber to reduce interference. The preamplifiers may be powered by rechargeable lithium polymer batteries with a DC voltage regulator to reduce electrical noise. In some cases, the ultrasonic detector array may include a printed circuit board (PCB) (e.g., a ring-shaped PCB) and the integrated pre-amplifiers are coupled to the PCB to receive ultrasonic signals from the ultrasonic receivers connected to the PCB.

During an example of a full imaging operation of UST system 100 shown in FIG. 1, the ultrasonic transmitter 114 is moved or rotated (depicted by a curved arrow) around a subject 11 (e.g., a human) being imaged. In one implementation, the ultrasonic detector array assembly 110 includes a motor such as a stepper motor configured to move the ultrasonic transmitter 114 along a line, an arc, or a circle. In one case, the ultrasonic array assembly 110 also includes a (motorized) rotational shaft and a gear or other component rotatably coupled to the rotational shaft. In this case, the ultrasonic transmitter 114 is mounted onto the mechanical gear or other component that rotates around the subject 11. During the full imaging operation, the motor rotates the rotation shaft, which rotates the gear, moving the ultrasonic transmitter 114 in, for example, a circular path around subject 11.

During various imaging operations, the ultrasonic detector array 112 is acoustically coupled with subject 11 to detect ultrasound signals transmitted, reflected and/or scattered by the subject 11. In one implementation, the UST system 100 may include a tank of acoustic coupling medium (e.g., water or acoustic gel) and the subject 11 may be at least partially submerged in the medium during image acquisition. For example, the tank may be a water immersion tank. In another implementation, one or more packages or containers of acoustic coupling medium (e.g., water packs or inflatable water bags) may be placed in contact with subject 11 and the ultrasonic receivers 113. It would be understood that when the UST system 100 is not in operation, the subject 11 may not be present (denoted by dashed line).

In one embodiment, the UST system 100 may also include one or more translational stages upon which the ultrasonic detector array 112 is mounted. For example, the ultrasonic detector array 112 may be mounted on two vertical motor stages to adjust the ultrasonic detector array 112 to different locations along the z-axis (heights) in a water immersion tank. The water in the immersion tank acts as an acoustic coupling medium between the subject 11 and the ultrasonic receivers 113. During the full imaging operation, the ultrasonic detector array 112 may be moved to different z-locations. For example, scans may be performed at vertical intervals such as 1 cm intervals.

The ultrasonic detector array 112 includes a plurality of integrated pre-amplifiers 118 (e.g., low-noise parallel preamplifiers) coupled to the ultrasonic receivers 113. In one implementation, ultrasonic detector array 112 includes a printed circuit board (PCB) (e.g., a ring-shaped PCB) and the integrated pre-amplifiers 118 are coupled to the PCB to receive ultrasonic signals from the ultrasonic receivers 113 connected to the PCB. During imaging operations, the receiver channels from the plurality of ultrasonic receivers 113 may be amplified (boosted) by the pre-amplifiers 118, for example, in one-to-one correspondence. In another implementation, the pre-amplifiers 118 are separate components from the ultrasonic detector array 112. In this case, the pre-amplifiers 118 are electrically connected to the ultrasonic receivers 113.

In various examples of imaging systems described herein, the imaging system includes one or more DAQs connected to pre-amplifiers that are connected to the ultrasonic receivers of the ultrasonic detector array to digitize and record ultrasound signals detected by the receivers during operation. An example of a suitable DAQ that can be employed is a DAQ made by Photosound Legion. The one or more DAQs record ultrasound signals according to a sampling frequency. In one aspect, the sampling frequency is about 5 MHz. In another aspect, the sampling frequency is about 40 MHz. According to various aspects, the sampling frequency is in a range from about 4 MHz to about 100 MHz. In one aspect, the one or more DAQs may be located within shielded enclosures. In an example of a parallel digitizing implementation, each pre-amplifier is connected to one ultrasonic receiver and each DAQ digitizer is connected to one pre-amplifier. In some cases, an imaging system also includes a plurality of low-pass filters connected to the ultrasonic receivers to receive ultrasonic signals and connected to he DAQ(s) to avoid aliasing when sampling. As used herein, “connected to” refers to direct coupling to the element or indirect connection made through one or more electrical connectors or other electronic devices. In some cases, the connection may be in a wireless form.

In FIG. 1, UST system 100 includes one or more data acquisition modules (DAQs) 130 and a plurality of low-pass filters 132 connected to the plurality of pre-amplifiers 118 and with a plurality of digitizers of the DAQ(s) 130. An example of a suitable low-pass filter 132 is a low-pass filter with cutoff frequency (fc) of about 2.2 MHz or about 2.0 MHz. The low-pass filters 132 may be employed to avoid aliasing when sampling. The DAQs 130 may be located within shielded enclosures. FIG. 1 depicts a parallel digitizing implementation where amplified parallel analog acoustic signals from a plurality of pre-amplifiers 118 are received by a respective plurality of low-pass filters 132 in one-to-one correspondence and each low-pass filter 132 is connected to one DAQ digitizer such that parallel analog acoustic signals from the low-pass filters 132 may be digitized in parallel by the DAQ(s) 130. For example, all channels may be low-pass filtered (fc=2 MHZ) and digitized in parallel at, e.g. at 5 MSPS. In another implementation, the low-pass filters 132 may be omitted.

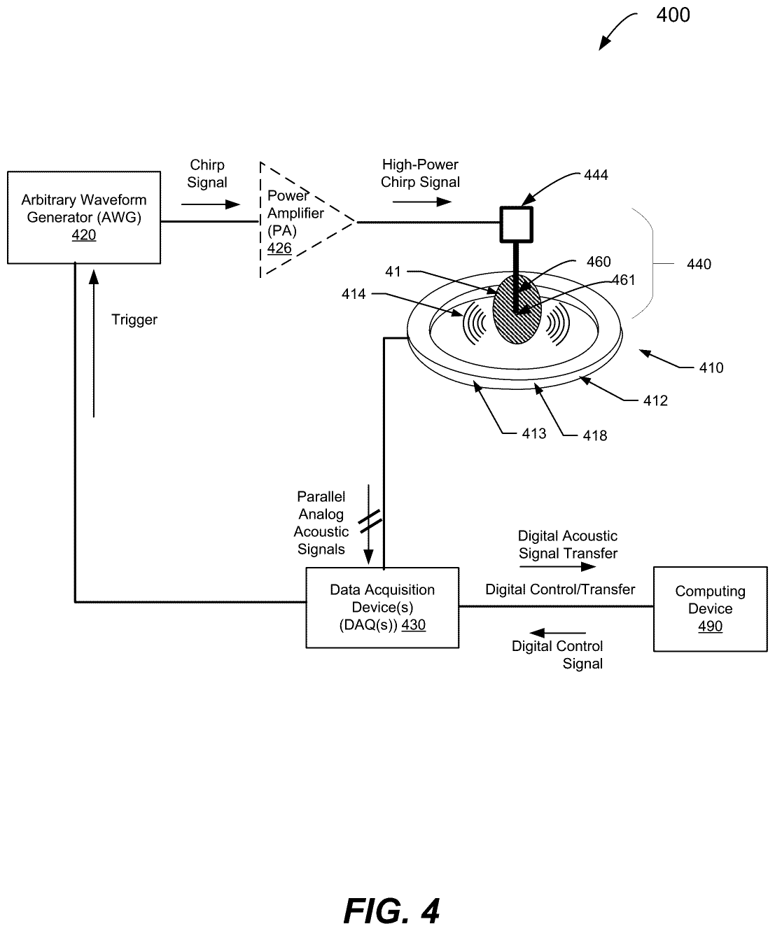

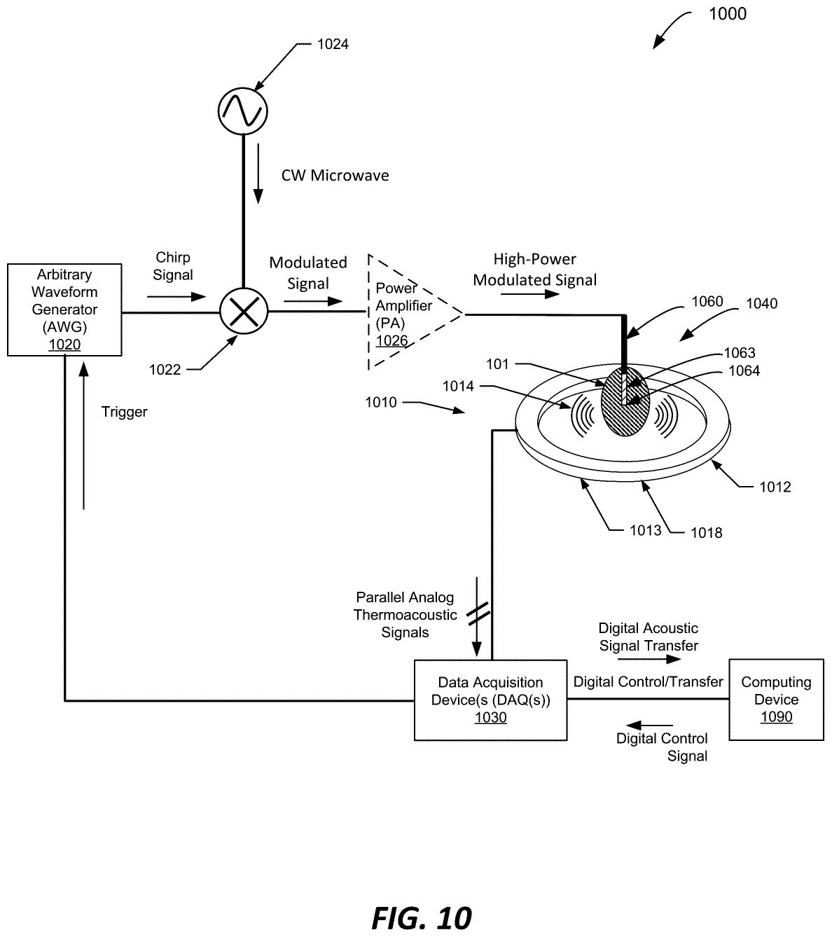

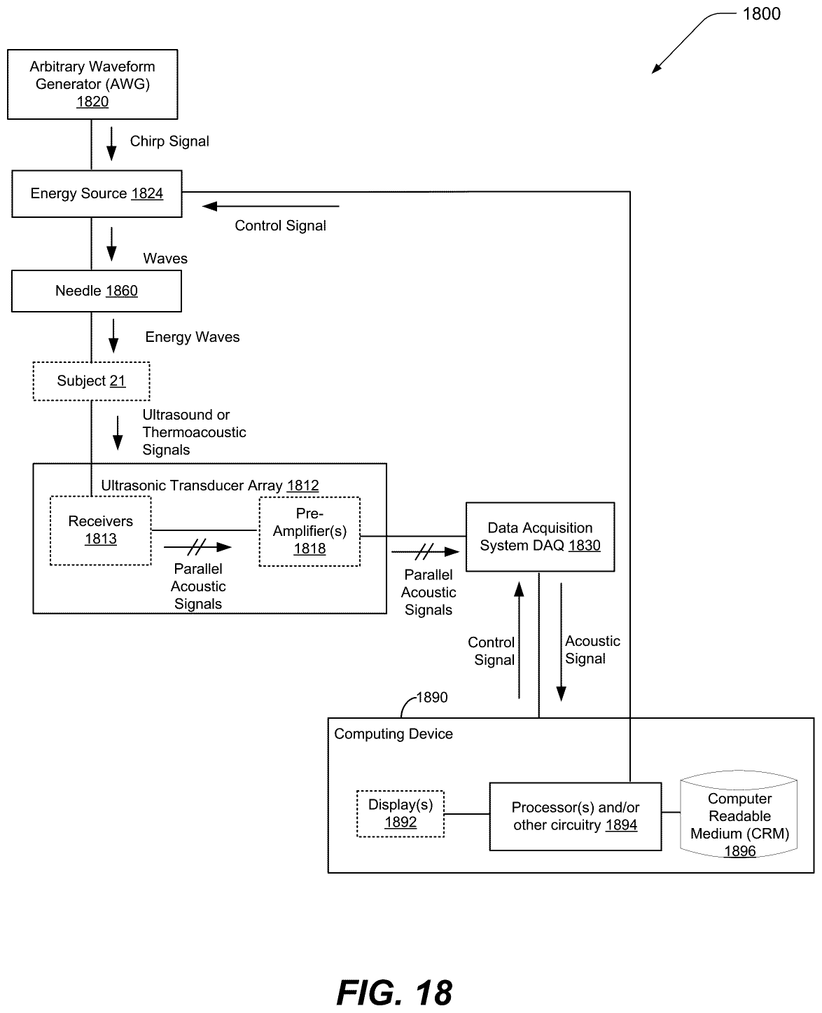

In certain implementations, to enhance signal-to-noise ratio (SNR) a chirp signal is used to excite an energy source such as an acoustic transmitter or is used to modulate an energy source such as a continuous wave source. In various imaging systems described herein, the imaging system include an arbitrary waveform generator (AWG) (e.g., AWG 120 in FIG. 1, AWG 420 in FIG. 4, AWG 1020 in FIG. 10, and AWG 1820 in FIG. 18) to generate a chirp signal sweeping over a customized frequency range and duration. An example of a suitable AWG is the SFG2042X arbitrary waveform generator made by Siglent. In embodiments with biopsy needle localization, for example, a chirp signal may be used to excite an acoustic transmitter (e.g., acoustic transmitter 444 in FIG. 4). In one case, to enhance SNR without exceeding the mechanical index (MI) safety standard, a 400 μs long linear chirp signal spanning a frequency range of 0.30-2.0 MHz may be used to excite an ultrasonic transmitter. According to another aspect, a linear chirp signal spanning a frequency range of 0.05 to 1.0 MHz and with a duration in a range of 50-500 μs can be used to excite an ultrasonic transmitter. According to another aspect, a quadratic chirp signal spanning a frequency range of 0.05 to 2.0 MHz and with a duration in a range of 50-500 μs can be used to excite an ultrasonic transmitter. According to another aspect, a linear chirp signal spanning a frequency range of 1.0 to 5.0 MHz and with a duration in a range of 50-500 μs can be used to excite an ultrasonic transmitter. In embodiments with ablation needle monitoring, a chirp signal may be used to modulate a continuous wave (CW) source (e.g., microwave source 1024 in FIG. 10). According to another aspect, a linear chirp signal spanning a frequency range of 0.15 to 2.0 MHz and with a duration in a range of 50-400 μs can be used to excite a transducer coupled to a biopsy needle. According to another aspect, a quadratic chirp signal spanning a frequency range 0.15 to 2.0 MHz and with a duration in a range of 50-400 μs can be used to excite a transducer coupled to a biopsy needle.

In FIG. 1, UST system 100 includes an arbitrary waveform generator (AWG) 120, an optional (denoted by dashed line) power amplifier 126 connected to the AWG 120, and an optional (denoted by dashed line) matching network (MN) 128 connected to the power amplifier 126 and the ultrasonic transmitter 114. The AWG 120 can provide a customized voltage such as a chirp signal sweeping over a customized frequency range and duration such as, e.g., a duration of about 400 μs, and a frequency in a range between 0.3 MHz and 2.0 MHz. Matching network 128 may include a circuit of inductors and capacitors for increasing coupling to ultrasonic transmitter 114. The power amplifier (PA) 126 can increase the power of the chirp signal to output a high-power chirp signal. To image deep into subject 11, the signal sensitivity may be enhanced by employing the power amplifier (PA) 126 and by exciting the ultrasonic transmitter 114 with the chirp waveform.

In certain implementations, an imaging system described herein (e.g., UST 100 in FIG. 1, biopsy needle localization system 400 in FIG. 4, needle ablation monitoring system 1000 in FIG. 10, and tomography imaging system 1800 in FIG. 18) may include electromagnetic (EM) shielding (e.g., a stainless steel plate) that encloses, or walls off, one or more electrical components of the system to block waves generated by the components from propagating to the ultrasonic detector array. For example, the DAQ(s) 130 in FIG. 1 may be located within shielded enclosures. In other implementations, the ultrasonic sensor array (ultrasonic detector array 112 in FIG. 1) may be enclosed within EM shielding. According to one aspect, EM shielding may be part of a housing within which one or more electrical components are disposed.

UST system 100 also includes a computing device 190 with one or more processors (e.g., a CPU, GPU or computer, analog and/or digital input/output connections, controller boards, etc.) and/or other circuitry 194, an optional (denoted by dashed line) display 192 connected to the processor(s) and/or other circuitry 194, and a computer readable media (CRM) 196 (e.g., a non-transitory computer readable media) connected to the processor(s) or other circuitry 194. Computing device 190 is connected to DAQ(s) 130 to receive ultrasonic data. Processor(s) and/or other circuitry 194 are connected to CRM 186 to store and/or retrieve data. The one or more processor(s) and/or other circuitry 194 are connected to optional display 192 for, e.g., displaying one or more images. Although not shown, computing device 190 may also include a user input device for receiving data from an operator of UST system 100. The computing device 190 may be, for example, a personal computer, an embedded computer, a single board computer (e.g., Raspberry Pi or similar), a portable computation device (e.g., tablet), or any other computation device or system of devices capable of performing the functions described herein. Optionally, computing device 190 may be connected to a controller to send control signals with control and synchronization data.

In one aspect, one or more processors and/or other circuitry 194 may execute instructions stored on the CRM 186 to perform one or more operations of a UST method. For example, processor(s) 184 and/or other circuitry 194 may execute instructions for: 1) communicating control signals to one or more components of UST system 100 and 2) reconstructing one or more cross-sectional images of the subject or portions thereof. In one implementation, ultrasound signals detected by the ultrasonic sensor array 112 may be streamed to computing device 190 by DAQ(s) 130. After which, computing device 190 may reconstruct a sequence of ultrasound images.

The connections between components of UST system 100 may be in wired and/or wireless form. One or more of the electrical connections between components of a UST system may be able to provide power in addition to communicate signals. During operation, digitized data may be first stored in an onboard buffer, and then transferred to the computing device of a UST system, e.g., through a universal serial bus 2.0.

In some embodiments, an imaging system described herein includes one or more communication interfaces (e.g., a universal serial bus (USB) interface). Communication interfaces can be used, for example, to connect various peripherals and input/output (I/O) devices such as a wired keyboard or mouse or to connect a dongle for use in wirelessly connecting various wireless-enabled peripherals. Such additional interfaces also can include serial interfaces such as, for example, an interface to connect to a ribbon cable. It should also be appreciated that the various system components can be electrically coupled to communicate with various components over one or more of a variety of suitable interfaces and cables such as, for example, USB interfaces and cables, ribbon cables, Ethernet cables, among other suitable interfaces and cables.

During an exemplary full imaging operation of the UST system 100 shown in FIG. 1, the subject 11 may be placed in contact with an acoustic medium during each scanning operation (scan) in which the ultrasonic transmitter 114 is rotated around the subject 11. For instance, a human subject may be seated in a water immersion tank during each scanning operation. In one example, the duration of each scanning operation may be 10 seconds. In other examples, other durations may be used such as 1-20 seconds. During each scanning operation, the ultrasonic detector array 112 detects ultrasonic signals, which can be used to generate cross-sectional images. Cross-sectional images can be created at different instances of time to capture multiple frames of, for example, a video. Some examples of cross-sectional images that can be acquired include isotropic images of reflectivity, speed of sound, and attenuation profiles.

UST system 100 can operate in both reflection and transmission modes simultaneously. For example, received signals on the transmitter side are used for reflection mode image reconstruction, while received signals opposite the transmitter are used for transmission mode image reconstruction. As the transmitter rotates, different receiver channels are used for reflection and transmission mode image reconstruction.

The reflected and transmitted signals can be distinguished by which receiver channels they occur on relative to the transmitter position. For instance, at the starting transmitter position, the channels on the half of the detector array on the transmitter side may be used for reflection mode, and the channels opposite the transmitter ma used for transmission mode. As the transmitter rotates, different receiver channels are used to correspond to reflection and transmission.

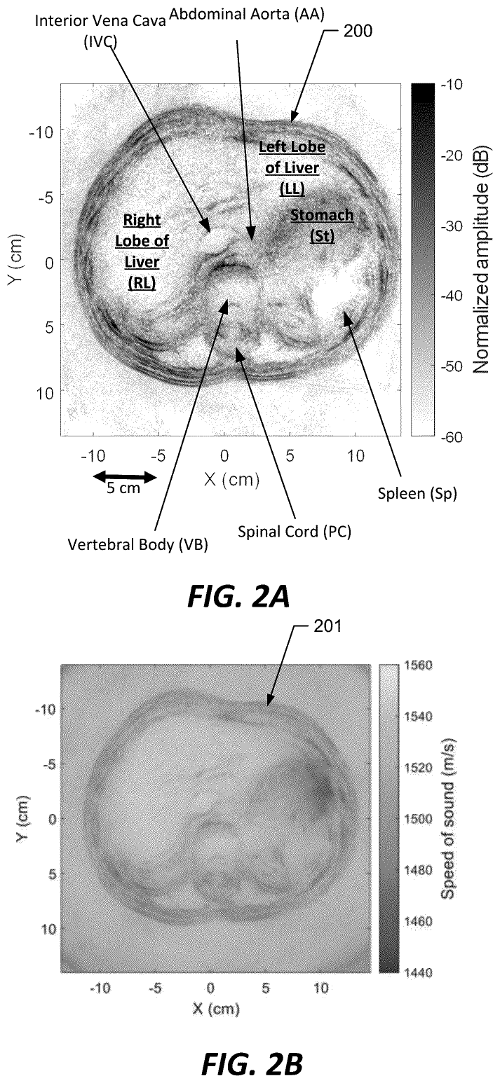

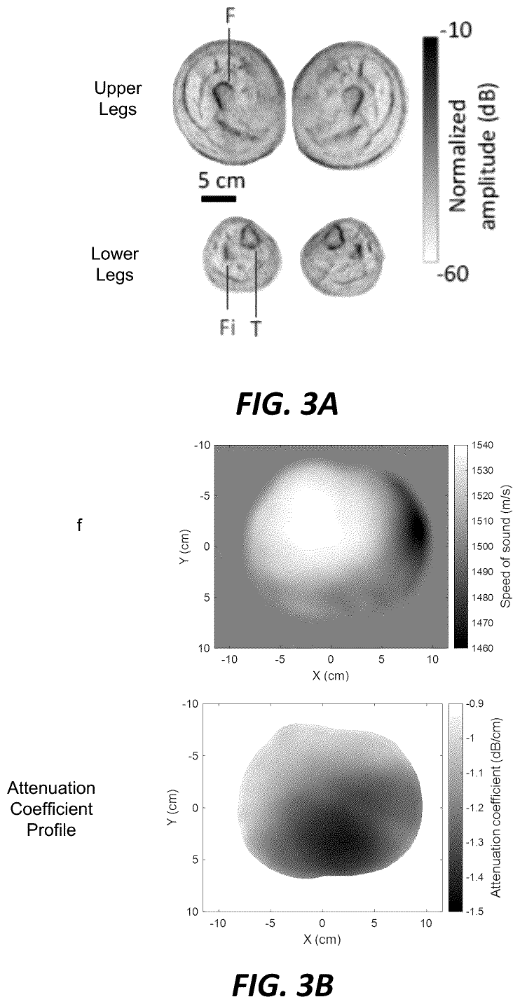

Example cross-sectional tomographic images of a human abdomen acquired using the UST system 100 in FIG. 1 are shown in FIGS. 2A and 2B, where several organs (e.g., liver, stomach, spleen) and features (e.g., abdominal aorta, interior vena cava, vertebral body, spinal cord, left and right lobes of liver, etc.) can be observed. FIG. 2A is an example of a reflection-mode image 200 of a human abdomen resulting from a full imaging operation of the UST system 100 shown in FIG. 1, according to an embodiment. Despite the presence of bone and air pockets in the body, the geometry of the UST system 100 enables imaging of regions deep in the body. Due to the lower acoustic frequency than typical probe-based ultrasonography uses, the images correspond primarily to reflections from tissue boundaries rather than from scattering within tissues. FIG. 2B is an example of an image of a speed of sound profile 201 overlaid on the reflectivity image 200 from FIG. 2A, according to an embodiment. The image 201 in FIG. 2B also resulted from such a full imaging operation of the UST system 100 shown in FIG. 1. FIG. 3A depicts reflection-mode images of the legs of a human body acquired using the UST system 100 in FIG. 1, according to an embodiment. In the upper legs, the femur (F), surrounding muscle groups, and adipose boundaries are observed. The tibia (T) and fibula (Fi) are visualized in the lower legs as well as adipose boundaries. Signals transmitted through the body can be used to reconstruct profiles of the speed of sound and attenuation coefficient profiles. FIG. 3B depicts speed of sound and attenuation coefficient profiles of a human abdomen acquired using the UST system 100 in FIG. 1, according to an embodiment. The reflectivity images could be used to help guide the biopsy or ablation needle to the appropriate region inside the body. The needle would be observed with respect to the internal features. The speed of sound and attenuation profiles could be used during biopsy/ablation needle image reconstruction to account for acoustic heterogeneity and to recover more accurate needle images.

In various embodiments, imaging systems include acoustic transmitters that emit acoustic waves. According to one aspect, the acoustic frequency used by the acoustic transmitters may span a range of 0.3 to 2.0 MHz. According to another aspect, the acoustic frequency used by the acoustic transmitters may span a range of 0.05 to 2.0 MHz. According to another aspect, the acoustic frequency used by the acoustic transmitters may span a range of 1.0 to 5.0 MHz. According to another aspect, the acoustic frequency used by the acoustic transmitters may span a range of 0.05 to 1.0 MHZ.

To image deep into the subject 11, the signal sensitivity of UST 100 may be enhanced by employing the power amplifier 126 coupled to the preamplifiers 118 of the ultrasonic detector array 112 and by exciting the ultrasonic transmitter 114 with a chirp waveform. In comparison with conventional handheld ultrasonic probes, the UST 100 may reduce issues of acoustic shadowing from regions containing bone or air pockets by using full 360 degree viewing angles provided by the circular ultrasonic detector array. In comparison to MRI and other standard imaging modalities, such UST imaging may be a low-cost, safe, and convenient tool for screening and monitoring conditions.

An example of a combined UST and TAT system can be found in U.S. patent application Ser. No. 18/336,863, titled “THERMOACOUSTIC AND ULTRASOUND TOMOGRAPHY,” filed on Jun. 16, 2023, which is hereby incorporated by reference in its entirety and for all purposes. FIG. 10 depicts an example of a microwave ablation monitoring system 1000 that can implement TAT techniques.

### II. Biopsy Needle Localization

In clinical practice, when a region is suspected of being cancerous, a small sample of tissue may be collected using an inserted biopsy needle. To guide the positioning of the biopsy needle with respect to internal features, current methods use conventional ultrasound and X-ray CT techniques. However, current methods of ultrasound localization only function properly for relatively superficial targets like the breast, and require the biopsy needle to be approximately orthogonal to the imaging probe to provide sufficient backscatter. To improve ultrasound visibility, some methods use treatments like scoring or bubble-filled polymer coatings to generate more isotropic scattering. However, these approaches can increase the insertional friction of the needle. Current CT needle guidance may allow for whole-body localization, but it requires iterative positioning and can lead to harmful radiation to the patient.

Certain embodiments pertain to systems, devices, and methods for biopsy needle localization. In these biopsy needle localization techniques, ultrasound signals are coupled into a biopsy needle, which leads to acoustic scattering at the needle tip. These ultrasound signals can be detected from outside the subject with an ultrasonic detector array. The ultrasonic signals detected by the ultrasonic detector array can then be used to reconstruct an image at the needle tip to determine the location of the needle tip. This approach may enable fast, safe, and precise needle localization to a region within the whole subject (whole body). In some cases, UST techniques may be employed for whole-body ultrasound imaging to generate one or more cross-sectional UST images of the subject. For example, the ultrasonic transmitter 114 in FIG. 1 may be employed for a full imaging operation to acquire one or more cross-sectional UST images. The image of the needle tip can then be superimposed onto the cross-UST image to determine the location of the needle in vivo within the subject.

In certain embodiments, a biopsy needle localization apparatus includes a biopsy needle and an ultrasonic transmitter (transducer) acoustically coupled to the biopsy needle. The ultrasonic transmitter (transducer) may be acoustically coupled directly to (in direct contact with) the needle or via a separate acoustic coupler. In some cases, a handle or other portion of the body of the biopsy needle localization apparatus may function as a separate acoustic coupler. For example, the ultrasonic transmitter may be attached to a handle or other portion of the body that is acoustically coupled to the biopsy needle. During operation, acoustic waves from the ultrasonic transmitter are acoustically coupled into the biopsy needle, and acoustic waves propagate along the needle until they are scattered at the needle tip. The ultrasound signals can be detected by an ultrasonic detector array and used to recover the location of the needle tip. In some cases, the location of the needle tip can be recovered at about 7 frames per second.

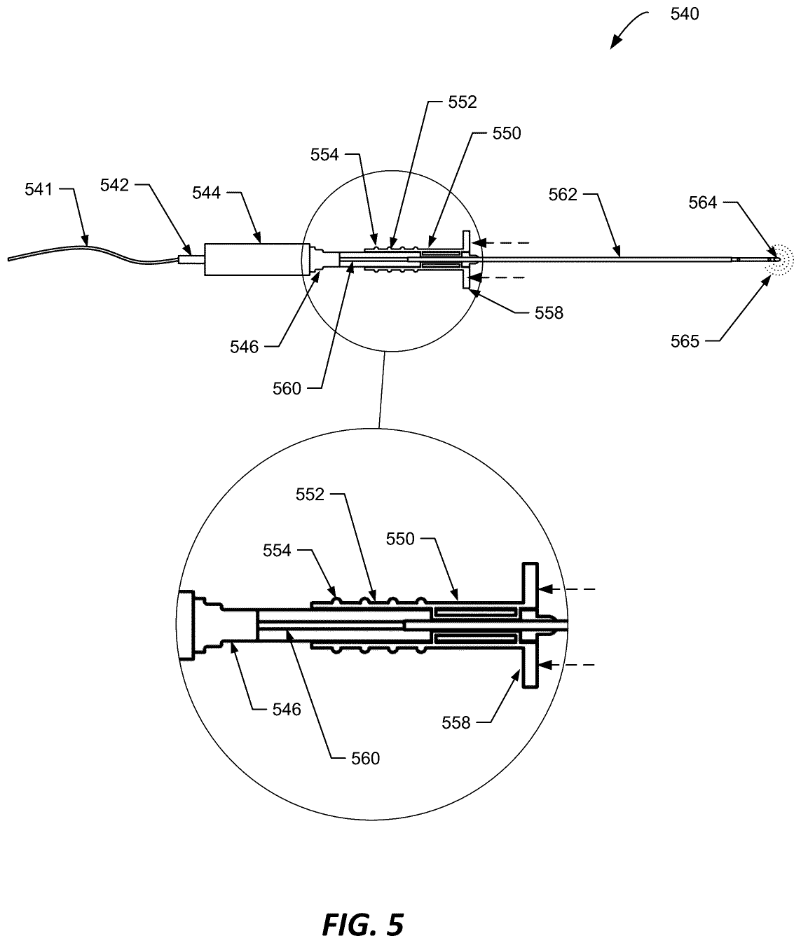

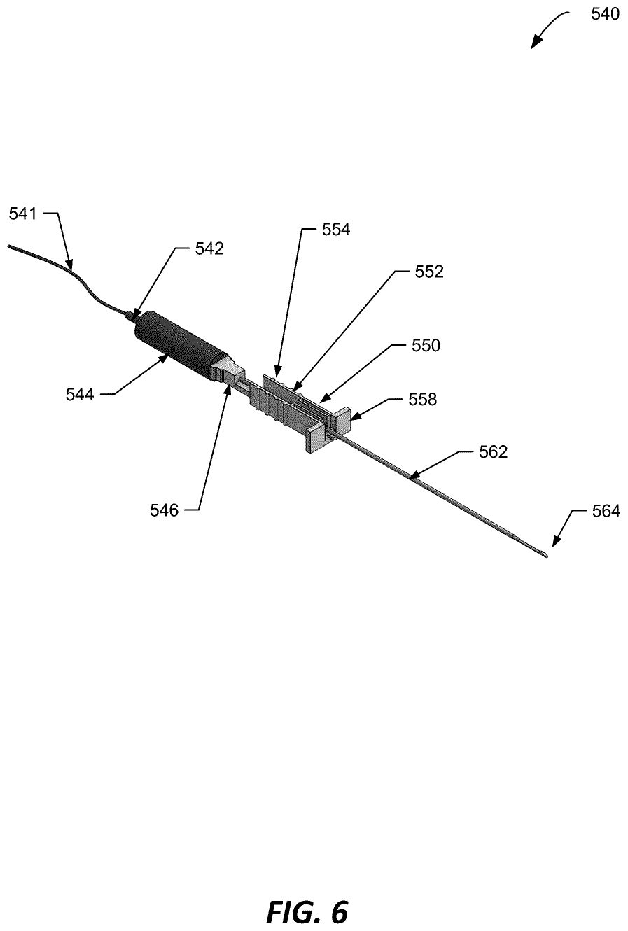

FIG. 4 depicts a block diagram of components of a biopsy needle localization system 400, according to various implementations. Biopsy needle localization system 400 includes a biopsy needle localization device 440 having an acoustic transmitter 444 coupled to a biopsy needle 460 having a needle tip 461. The block diagram shows the flow for biopsy needle localization using acoustic coupling to the biopsy needle 460 and parallel detection with an ultrasonic detector array 412. The illustrated example in FIG. 4 is shown at an instant in time when needle tip 461 is inserted into subject 41. It would be understood that subject 41 may not be present at other times such as when the system is not in operation. Biopsy needle localization device 540 described with reference to FIGS. 5 and 6 is an example of an implementation of the biopsy needle localization device 440 shown in FIG. 4.

Biopsy needle localization system 400 also includes the ultrasonic detector array assembly 410 having an ultrasonic detector array 412 with a plurality of ultrasonic receivers 413 and a plurality of integral pre-amplifiers 418 connected (e.g., one-to-one correspondence) to the ultrasonic receivers 413 to receive parallel analog acoustic signals. Biopsy needle localization system 400 also includes one or more data acquisition devices (DAQs) 430 with digitizers connected (e.g., one-to-one correspondence) to the ultrasonic receivers 413 to receive parallel analog acoustic signals. Biopsy needle localization system 400 also includes an arbitrary waveform generator (AWG) 420 for generating a chip signal and an optional (denoted by dashed line) power amplifier 426 connected to the AWG 420 to receive the chirp waveform and connected to the acoustic transmitter 444 to provide a high-power chirp signal to excite the acoustic transmitter 444 to generate an acoustic signal.

The acoustic signal is coupled into the biopsy needle 460 and propagates along the needle to generate acoustic waves at tip 461 which are transmitted, reflected and/or scattered by the subject 41 to generate acoustic signals 414. These acoustic signals 414 can be detected by the ultrasonic receivers 413 of ultrasonic detector array 412. The detected ultrasonic signals are then digitized and recorded by the DAQ(s) 430.

Biopsy needle localization system 400 also includes a computing device 490 and one or more DAQs 430 connected to the computing device 490 to receive control signals and/or send digital acoustic signal data. The DAQ(s) 430 are also connected to the AWG 420 to send signals to trigger chirp signals. For the sake of brevity, the prior discussion of the ultrasonic detector array assembly 110, ultrasonic detector array 112, ultrasonic receivers 113, pre-amplifiers 118, data acquisition devices (DAQs) 130, arbitrary waveform generator 120, power amplifier 126, and computing device 190 with regards to FIG. 1 may be assumed to be equally applicable, unless indicated otherwise, to similar or analogous counterparts of those elements in FIG. 4 that share the same last two digits in their respective callouts as in FIG. 1. Also, biopsy needle localization system 400 may include one or more components (e.g., stepper motor) for moving ultrasonic detector array 412 to different elevational levels for scans.

In certain embodiments, a biopsy needle localization system may include the ultrasonic transmitter 114 and other components of the UST system 100 in FIG. 1 to enable a whole-body imaging procedure. For example, in one embodiment, biopsy needle localization system 400 in FIG. 4 may include the ultrasonic transmitter 114, components used to rotate the ultrasonic transmitter 114, the matching network 128, and the low-pass filters 132 described with respect to FIG. 1 to enable acquiring cross-sectional images of the subject 41 using a UST technique. In this implementation, ultrasonic transmitter 114 from FIG. 1 may be coupled to the PA 426 to receive a high-power chirp signal.

During imaging operations such as a needle localization procedure or a full imaging procedure, the ultrasonic detector array 412 is acoustically coupled with the subject 41 being imaged to detect transmitted, reflected and/or scattered ultrasound signals. For example, the biopsy needle localization system 400 may include a tank of acoustic coupling medium (e.g., a water immersion tank) and the subject 41 may be at least partially submerged in the medium during image acquisition. In another implementation, one or more packages or containers of acoustic coupling medium (e.g., water packs) may be placed in contact with the subject 41 and the ultrasonic receivers 413.

In FIG. 4, biopsy needle localization system 400 includes a plurality of pre-amplifiers 418 that are integral to the ultrasonic detector array 412. In another implementation, the pre-amplifiers 418 may be separate from the ultrasonic detector array 412.

In FIG. 4, biopsy needle localization system 400 also includes one or more data acquisition modules (DAQs) 130 with a plurality of integrated digitizers that are in electrical communication in one-to-one correspondence with a respective plurality of pre-amplifiers 418. The illustrated example depicts a parallel digitizing implementation where amplified parallel analog acoustic signals from the pre-amplifiers 418 are received by a respective plurality of DAQ digitizers such that parallel analog acoustic signals may be digitized in parallel by the DAQ(s) 430.

Biopsy needle localization system 400 also includes an arbitrary waveform generator (AWG) 420 for providing a voltage of a chirp signal sweeping over a customized frequency range and duration. An example of a suitable frequency range and duration is about 400 μs, and a frequency in a range between 0.3 MHz and 2.0 MHz. Other suitable frequency ranges and durations are described in the previous section.

Biopsy needle localization system 400 also includes a computing device 490. The computing device 490 includes one or more processors and/or other circuitry, an optional display connected to the processor(s), and a computer readable media (CRM) connected to the processor(s) or other circuitry. Computing device 490 is connected to DAQ(s) 430 to receive ultrasound data. Processor(s) and/or other circuitry are connected to CRM to store and/or retrieve data. The one or more processor(s) and/or other circuitry are connected to optional display for, e.g., displaying one or more images. Although not shown, computing device 490 may also include a user input device for receiving data from an operator of biopsy needle localization system 400. The computing device 490 may be, for example, a personal computer, an embedded computer, a single board computer (e.g., Raspberry Pi or similar), a portable computation device (e.g., tablet), or any other computation device or system of devices capable of performing the functions described herein. Optionally, computing device 490 may be in communication with a controller to send control signals with control and synchronization data for synchronizing the functions of the system components.

In one aspect, the one or more processors of computing device 490 and/or other circuitry may execute instructions stored on the CRM to perform one or more imaging operations such as a biopsy needle localization operation. For example, processor(s) and/or other circuitry may execute instructions for: 1) communicating control signals to one or more components of biopsy needle localization system 400 to excite the acoustic transmitter 444 and record acoustic signals during a needle location procedure and/or to perform scanning operations to move an ultrasonic transmitter (e.g., ultrasonic transmitter 114 in FIG. 1) around the subject 41 and record acoustic signals during a full imaging UST procedure, 2) determining the location of the tip 461 of the biopsy needle 460 based on the recorded ultrasound data, 3) reconstructing one or more cross-sectional images of the subject or portions thereof based on the recorded ultrasound data, and/or 4) superimposing the location of the tip 461 onto a cross-sectional image. In one implementation, ultrasound signals detected by the ultrasonic detector array 412 may be streamed to computing device 490 by DAQ(s) 430. After which, computing device 490 may reconstruct a sequence (frames) of ultrasound images.

The electrical communication between components of biopsy needle localization system 400 may be in wired and/or wireless form. One or more of the electrical communications between components of a biopsy needle localization system 400 may be able to provide power in addition to communicate signals. During operation, digitized data may be first stored in an onboard buffer, and then transferred to the computing device 490 of biopsy needle localization system 400, e.g., through a universal serial bus 2.0.

During an exemplary needle localization procedure, subject 41 is placed in contact with an acoustic medium (e.g., submerged in a water immersion tank) and the AWG 420 is activated to excite the acoustic transmitter 444 to generate a chirp waveform. The chirp signal from the AWG 420 may be amplified in power by PA 426 and the high-power chirp signal delivered to excite the acoustic transmitter 444 to generate acoustic signals at tip 461 of the biopsy needle 460 and the ultrasonic detector array 412 detects transmitted, reflected and/or scattered ultrasound signals 414. The DAQ(s) 430 digitizes and records the ultrasound signals and the computing device 490 determines the location of the biopsy needle 460 using the ultrasound data.

In the implementation of the biopsy needle localization system 400 with the ultrasonic transmitter 114, components used to rotate the ultrasonic transmitter 114, the matching network 128, and the low-pass filters 132 as described with respect to FIG. 1, biopsy needle localization system 400 may perform a whole body imaging procedure to acquire one or more cross-sectional images of the subject 41. During the whole body imaging procedure, the subject 41 is in contact with an acoustic medium during each scanning operation (scan) in which an ultrasonic transmitter 114 is rotated around the subject 41. During each scanning operation, the ultrasonic detector array 416 detects ultrasonic signals, which can be used to generate the one or more cross-sectional images. Cross-sectional images can be created at different instances of time to capture multiple frames of a video. Some examples of cross-sectional images that can be acquired include isotropic images of reflectivity, speed of sound, and attenuation profiles.

### Examples of Biopsy Needle Localization Devices

In various embodiments, a biopsy needle localization apparatus includes an ultrasonic transmitter (transducer) acoustically coupled to a biopsy needle via a separate acoustic coupler or via direct contact. An arbitrary waveform generator connected to the ultrasonic transmitter generates a voltage with a chirp signal to excite the ultrasonic transmitter. The acoustic waves are coupled into the needle, and they propagate along the needle until they are scattered at the needle tip. These signals may be detected by the ultrasonic detector array and used reconstruct images at the needle tip at, for example, about 7 frames per second.

FIG. 5 depicts a cross-sectional view of a biopsy needle localization device 540, according to embodiments. FIG. 5 also includes an expanded view of a portion of the biopsy needle localization device 540. FIG. 6 depicts an isometric view of the biopsy needle localization device 540 in FIG. 5.

Biopsy needle localization device 540 includes a biopsy needle 560 acoustically coupled to an acoustic transmitter 544 to enable needle tip localization within a subject such as a human body. The biopsy needle 560 may be a hollow needle that can be used to be inserted into a subject and used to extract tissue and fluid. In one example, the biopsy needle 560 is a solid stainless steel core (e.g., 1.5 mm diameter) needle. Biopsy needle localization device 540 also includes a body 550 with a handle 552 (e.g., a plastic handle) having a plurality of gripper protrusions 554 along an outer surface. The body 550 is generally cylindrical in shape. Other shapes may be used in other implementations. Biopsy needle localization device 540 also includes a hollow sheath or sleeve 562. The biopsy needle 560 and the hollow sheath or sleeve 562 are sized and aligned such that the biopsy needle 560 can translate within the hollow sheath or sleeve 562. The biopsy needle 560 includes a retractable needle tip 564 that can be used for acoustic scattering in a needle localization procedure and for tissue extraction. The body 550 also includes a flange 558 attached to the hollow sheath or sleeve 562. Pressure (depicted as dashed arrows) applied to the flange 558 causes the retractable needle tip 564 to extend out of the distal end of the hollow sheath or sleeve 562 as depicted in the illustration. After tissue extraction, the retractable needle tip 564 may be retracted back into the hollow sheath or sleeve 562.

In various embodiments, biopsy needle localization apparatus includes an ultrasonic transmitter and a separate coupling interface (acoustic coupler). The ultrasonic transmitter is an acoustic piezoelectric transmitter. The acoustic coupler provides acoustically coupling between the ultrasonic transmitter and the biopsy needle to propagate acoustic waves to the biopsy needle. These acoustic signals are propagated down the needle similar to an acoustic wave guide, and the signals are scattered isotropically at the needle tip. The acoustic coupler may be made of any suitable acoustic material such as silicone, propylene glycol, epoxy, or polyurethane, and may be of any suitable shape. In some cases, the acoustic coupler includes one or more components acoustically coupled together. In other embodiments, the biopsy needle localization apparatus may include an ultrasonic transmitter that is directly bonded to the biopsy needle using, for example, a high acoustic impedance adhesive.

In FIG. 5, biopsy needle localization device 540 includes a separate acoustic coupling interface (acoustic coupler) 546 between the biopsy needle 560 and acoustic transmitter 544. Biopsy needle localization device 540 also includes a cable 541 with an electrical connector 542 at a proximal end for connection to acoustic transmitter 544. The distal end of the cable 541 is connected to a PA (e.g., PA 426 in FIG. 4) or with AWG (e.g., AWG 420) in FIG. 4 to receive a chirp signal.

To improve SNR, a chirp signal is used to excite the acoustic transmitter 544 to generate acoustic waves (emissions) 565 at the retractable needle tip 564. The scattered signals from retractable needle tip 564 are detected by an ultrasonic detector array (e.g., ultrasonic detector array 412 in FIG. 4). The signals detected by the ultrasonic detector array may be cross correlated with a water chirp response.

FIG. 7A depicts an isometric view of components of a biopsy needle localization system 700, according to embodiments. Biopsy needle localization system 700 includes the components of the biopsy needle localization system 400 shown in FIG. 4 and components of the biopsy needle localization device 540 described with reference to FIGS. 5 and 6. Biopsy needle localization system 700 also includes a plurality of conduits 734 within which wiring (e.g., one or more cables) passes between ultrasonic detector array 412 and the DAQs 430 and/or other system components. The distal end of the cable 541 is connected to the PA 426 and/or AWG 420.

FIG. 7B depicts a cross-sectional view of an acoustic receiver 702 of an acoustic detector array, according to an embodiment. The acoustic receiver 702 includes a piezoelectric element capacitively coupled to copper cladded polyimide electrodes by bonding with high-strength epoxy. In one example, the piezoelectric element is a 1 mm thick, 3 mm×10 mm gold coated piezoelectric polymer (e.g., PVDF-TrFE made by PolyK Technologies LLC). A continuous copper cladded polyimide electrode is used for the ground reference. The electrodes are then directly connected to parallel preamplifiers implemented on annular printed circuit boards of an acoustic detector array. The preamplifiers provide 15 dB voltage gain with 100 kΩ input impedance.

### Experimental Data

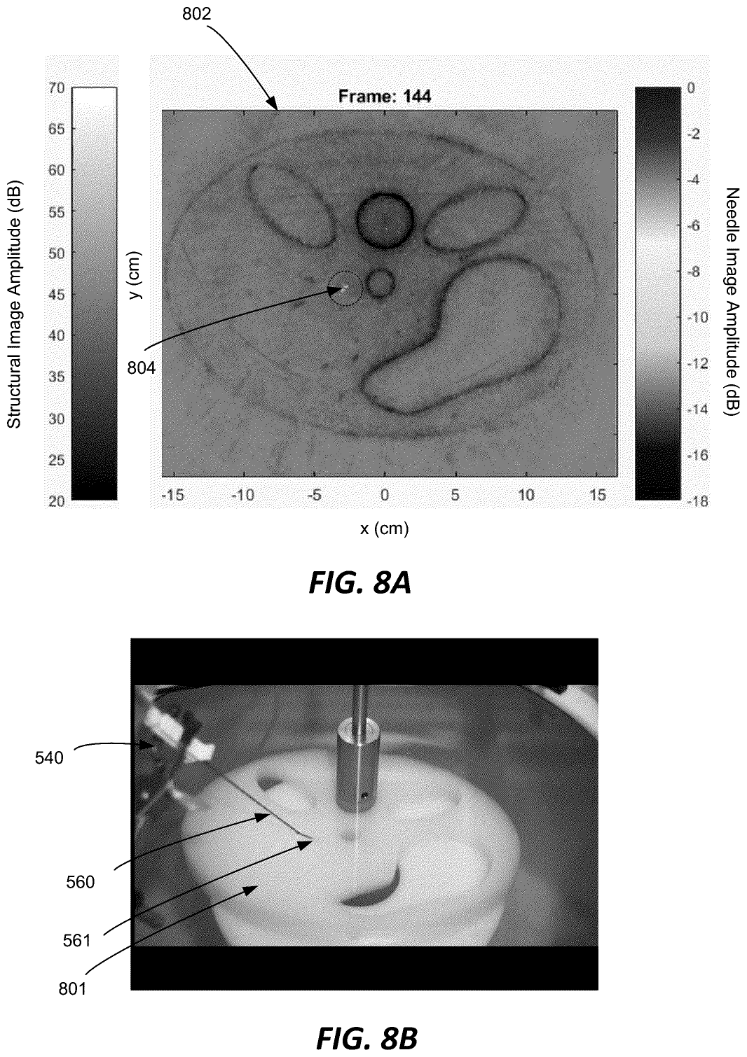

In one embodiment, a biopsy needle localization system includes the components of the biopsy needle localization system 400 as shown in FIG. 4, a single-element rotating ultrasonic transmitter (e.g., ultrasonic transmitter 114 in FIG. 1), a gear and a rotational shaft upon which the rotating ultrasonic transmitter is mounted, a stepper motor coupled to the gear to rotate ultrasonic transmitter, a matching network (e.g., matching network 128) in electrical communication between a power amplifier 426 and the rotating ultrasonic transmitter, low-pass-filters (e.g., low-pass filters 132 in FIG. 1) in electrical communication between the integral pre-amplifiers of the ultrasonic detector array 412 and the DAQ(s) 430, and a biopsy needle localization device 440 with the components of the biopsy needle localization device 540 described with reference to FIGS. 5 and 6. The ultrasonic detector array 412 implemented is a circular array with 512 ultrasonic receivers. FIGS. 8A and 8B illustrate results from a biopsy needle localization procedure performed by the biopsy needle localization system of this embodiment. In this procedure, the acoustic signals detected by ultrasonic detector array 412 are used to recover the location of the biopsy needle tip 461 at about 7 frames per second.

FIG. 8A is an example image frame 802 (“frame 144”) from a video generated by this biopsy needle localization system where the biopsy needle 560 shown in FIGS. 5 and 6 is inserted into a human-scale agarose phantom, according to an embodiment. FIG. 8B is a photograph of the experimental setup of the biopsy needle localization system, according to an embodiment. The illustrated example shows a biopsy needle 560 being inserted into a human-scale agarose phantom 801. Frame 402 in FIG. 8A shows the location 804 of the biopsy needle tip 561 superimposed (overlapped) onto the structural image (frame) 802 of the human-scale agarose phantom 801. The structural image 802 was obtained with a UST full imaging procedure by rotating the ultrasonic transmitter 114 around the subject. The location 804 of biopsy needle tip 561 was determined using a needle localization procedure that included emitting acoustic signals from the biopsy needle tip 561.

### Biopsy Needle Localization Method

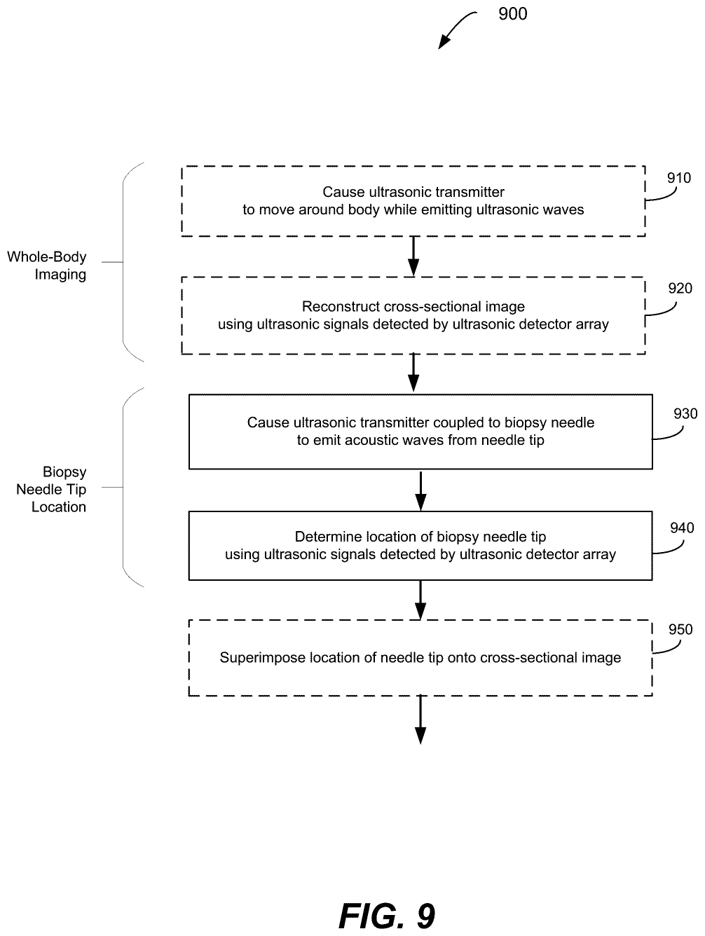

FIG. 9 depicts a flowchart 900 of a biopsy needle localization method, according to various embodiments. The biopsy needle localization method may be implemented by a biopsy needle localization system such as, for example, the biopsy needle localization system 400 in FIG. 4 or a biopsy needle location system that includes components of UST system 100 in FIG. 1 and components of the biopsy needle localization device 540 described with reference to FIGS. 5 and 6.

The biopsy needle localization method includes a biopsy needle location procedure (operations 930 and 940) for determining the location of the tip of a biopsy needle. Optionally, the biopsy needle localization method may also include a whole body imaging procedure (operations 910 and 920) that uses an UST technique to acquire one or more cross-sectional images. Also optionally, the biopsy needle localization method may include operation 950 that superimposes the image showing the location of the needle tip from the biopsy needle location procedure onto a cross-sectional image acquired by the whole body imaging procedure.

At optional (denoted by dashed line) operation 910, control signals are sent to components of the biopsy needle location system to cause the ultrasonic transmitter to move around (e.g., in a circular path around) the subject. For example, control signals may be sent to a stepper motor that rotates the rotation shaft, which rotates a gear on which the ultrasonic transmitter is mounted to move the ultrasonic transmitter around the subject. Control signals are also sent to the arbitrary waveform generator to emit a chirp signal to excite the ultrasonic transmitter during movement around the subject while there is ongoing data acquisition by an ultrasonic detector array. The ultrasonic detector array detects ultrasonic waves transmitted, reflected and/or scattered by the subject while the ultrasonic detector is emitting ultrasonic waves. One or more DAQs digitize and record the ultrasound signals detected by the ultrasonic detector array. Control signals may be sent automatically by a controller or computing device or may be initiated by manual input from an operator (e.g., turning on a device). In parallel digitizing implementations, parallel analog acoustic signals from a plurality of pre-amplifiers are received by a plurality of DAQ digitizers in one-to-one correspondence such that the parallel analog signals can be digitized in parallel.

At optional (denoted by dashed line) operation 920, ultrasonic image data is reconstructed from the raw ultrasonic signals detected over time by the ultrasonic detector array and recorded by the one or more DAQs. A plurality of images are reconstructed. Image reconstruction may include (i) reconstructing two-dimensional images over time and/or (ii) reconstructing volumetric three-dimensional images over time. Image reconstruction includes, at least in part, implementing an inverse reconstruction algorithm. Some examples of inverse reconstruction methods that can be used include: (i) forward-model-based iterative methods, (ii) time-reversal methods, and (iii) back projection methods. A 3D back projection method can be used to reconstruct a 3D volumetric image and a 2D back projection method can be used to reconstruct a 2D image. An example of a back projection method is the universal back-projection process described in U.S. patent application Ser. No. 17/090,752, titled “SPATIOTEMPORAL ANTIALIASING IN PHOTOACOUSTIC COMPUTED TOMOGRAPHY” and filed on Nov. 5, 2020, which is hereby incorporated by reference for this description. Another example of a back-projection method can be found in Anastasio, M. A. et al., “Half-time image reconstruction in thermoacoustic tomography,” IEEE Trans., Med. Imaging 24, pp 199-210 (2005). In another aspect, a dual-speed-of sound (dual-SOS) photoacoustic reconstruction process may be used. An example of a single-impulse panoramic photoacoustic computed tomography system that employs a dual-SOS photoacoustic reconstruction process is described in U.S. patent application 2019/0307334, titled “SINGLE-IMPULSE PANORAMIC PHOTOACOUSTIC COMPUTED TOMOGRAPHY” and filed on May 29, 2019. The detected signals may be cross-correlated with the chirp prior to reconstruction to recover a pulse-like representation.

In one embodiment, the biopsy needle localization method transmits the acoustic signal through the need and detects a scattered signal with the ultrasonic detection array. The detected ultrasound signal may be used to reconstruct an image of the needle tip and the image of the needle tip overlaid onto a UST image such as a reflection mode UST image.

At operation 930, control signals are sent to the arbitrary waveform generator to emit a chirp signal to excite the acoustic transmitter acoustically coupled to the biopsy needle to emit a scattered acoustic signal from the tip of the biopsy needle while there is ongoing data acquisition by the ultrasonic detector array (parallel detection). The ultrasonic detector array detects ultrasonic signals and one or more DAQs digitize and record the ultrasound signals. In parallel digitizing implementations, parallel analog acoustic signals from a plurality of pre-amplifiers are received by a plurality of DAQ digitizers in one-to-one correspondence such that the parallel analog signals can be digitized in parallel.

At operation 940, the location of the tip of the biopsy needle is determined using the ultrasonic signals detected by the ultrasonic detector array and recorded by the one or more DAQs. The location of the needle tip is determined by performing one-way back projection after accounting for the propagation time down the length of the needle. In one example, the location of the needle tip (e.g., needle's center response) may be determined based on the maximum image amplitude. The one-way back projection method accounts for the time delay down the length of the needle. In one embodiment, the biopsy needle localization method includes a calibration procedure that determines the propagation time along the biopsy needle. During the calibration procedure, the transmitter/transducer response is recovered using only acoustic medium (e.g., water) in the imaging domain. The transmitter/transducer response is then cross-correlated with the response with the subject present to recover a pulse-like representation. This may be done for the rotating transmitter where backscattered and transmitted signals are recorded in parallel while the transmitter is rotated. This may also be done to calibrate the transducer coupled to the biopsy needle.

At operation 950, an image with the location of the tip of the biopsy needle is superimposed on a two-dimensional whole body image. For example, the location of the needle may be overlaid on a UST reflectively image. In one aspect, transmission-mode speed of sound profiles may be used to improve the needle images by accounting for varying average speed of sound between the needle and each receiver channel. Example methods for doing this is are eikonal solvers or full-wave inversion.

Compared with other emerging techniques such as low-field MRI, whole-body UST techniques are faster (e.g., about 10 seconds per 2D slice) with comparable or finer resolution (e.g., about 1 mm), and it does not require a shielded room or magnet-compatible environment. Further, it is more portable, more open, and less noisy than MRI. Also, due to its magnet-free operation, UST techniques can be used for subjects with implants that are incompatible with MRI.

The needle localization techniques described herein may provide advantages over conventional CT guided techniques. For example, the location of the biopsy needle can be localized with respect to internal features without the use of ionizing radiation. Whereas CT guidance requires iterative needle positioning and imaging, needle localization techniques could enable real-time feedback. Also, the biopsy needle could be tracked with respect to internal features while performing a procedure that might deform tissues such as with procedures involving minimally invasive surgical robots.

### Acquisition Parameters

To enhance the signal-to-noise ratio (SNR) while limited by the mechanical index, a linear chirp signal versus time (t) can be used with a time varying frequency f(t)=fᵣt+f₀, where fᵣ=(f₁−f₀)/T is the linear chirp rate. In one example, f₀=0.3 MHz is the lower frequency, f₁=2.0 MHz is the upper frequency, and T=400 μs is the chirp duration. The transmitted frequencies are limited by the bandwidths of the transmitter and receivers. A maximal pulse duration was used given a maximal acquisition time of 800 μs, allowing for recovery of the roundtrip reflected signals over the entire field-of-view (FOV). The resulting transmitted chirp signal is:

$$x ( t ) = sin [ 2 π ( \frac{{f}_{r}}{2} {t}^{2} + {f}_{0} t ) ] \qquad ( Eqn . 1 )$$

Compared to a pulse with similar peak pressure, this results in an expected SNR gain of about \sqrt{T·B}, where B=f₁−f₀ is the acoustic bandwidth. In addition to the target, a scan was performed with only water in the imaging domain, resulting in recorded signals x_{w,i}(t) for each receiver element i in the acoustic detector array. This provides the response of each transducer to the chirp which is then cross-correlated with the target's chirp response x_{c,i}(t). The pulse response for the target signals x_{s,i}(t) is then recovered for each receiver element i as:

$${x}_{s , i} ( t ) = \frac{{x}_{w , i} ( t ) * {x}_{c , i} ( t )}{max [ {x}_{w , i} ( t ) * {x}_{w , i} ( t ) ]} \qquad ( Eqn . 2 )$$

where * denotes cross-correlation.

The maximum of the autocorrelation of x_{w,i}(t) is used to normalize to account for sensitivity variation in the receiver elements. In one example, the acoustic transmitter operates with a pulse repetition rate of 180 Hz. With a gear rotation time of 10 seconds, this results in 1800 transmitted pulses over a full circular scan around the subject (target).

### III. Needle Ablation Monitoring

Microwave needle ablation can be used to thermally treat cancerous regions to kill growing tumor cells. An ablating needle is positioned in a targeted area, and microwave signals are coupled through the needle to heat a cm-scale region around the tip. Conventional needle ablation techniques use prescribed durations and microwave power settings throughout the procedure. However, subject- and region-specific tissue differences can result in different heating rates with these conventional techniques.

Certain embodiments pertain to systems, devices, and methods for monitoring needle ablation. To better guide ablation treatment, these techniques generate and image thermoacoustic signals in a targeted area of the ablation. For example, by modulating microwave signals at acoustic frequencies being emitted at a radiating portion of the ablation needle, thermoacoustic signals can be generated in the heated tissue from thermoelastic expansion. These thermoacoustic signals are then detected from outside the body and can be used to obtain thermoacoustic images of a heating profile in tissue around the radiating portion. These techniques operate may use similar peak and average powers to those used in current clinical microwave ablation. Furthermore, thermoacoustic signals are advantageous for this ablation application since they are directly proportional to the heating rate in tissues. A similar approach can be used for sources of lower-frequency radiofrequency signals as well as using optical sources and ultrasound sources.

Some conventional clinical microwave ablation techniques employ about 100-300 W continuous wave microwaves over several minutes to heat cm-scale regions at a radiating portion near the tip of an inserted needle (probe). In these conventional techniques, heating times and microwave power are set as constant and are crudely determined without any feedback regarding the progress of the ablation.

The needle ablation monitoring techniques described herein take advantage of thermoacoustic signals generated when a modulated microwave source is used. In some cases, these techniques use similar average and peak powers to those by conventional clinical techniques. Thermoacoustic signals are appealing since they are directly proportional to the specific absorption rate in tissues, and they could therefore be used as a proxy for heating rate or tissue properties. These techniques can use the thermoacoustic signals generated to determine progress of the ablation to provide feedback during the ablation process.

In examples described herein, the needle ablation being implemented is microwave ablation. It would be understood that other types of ablation can be similarly performed using other sources such as other radio frequency sources and optical (light) sources to perform radio frequency ablation, optical ablation, ultrasonic ablation, etc.

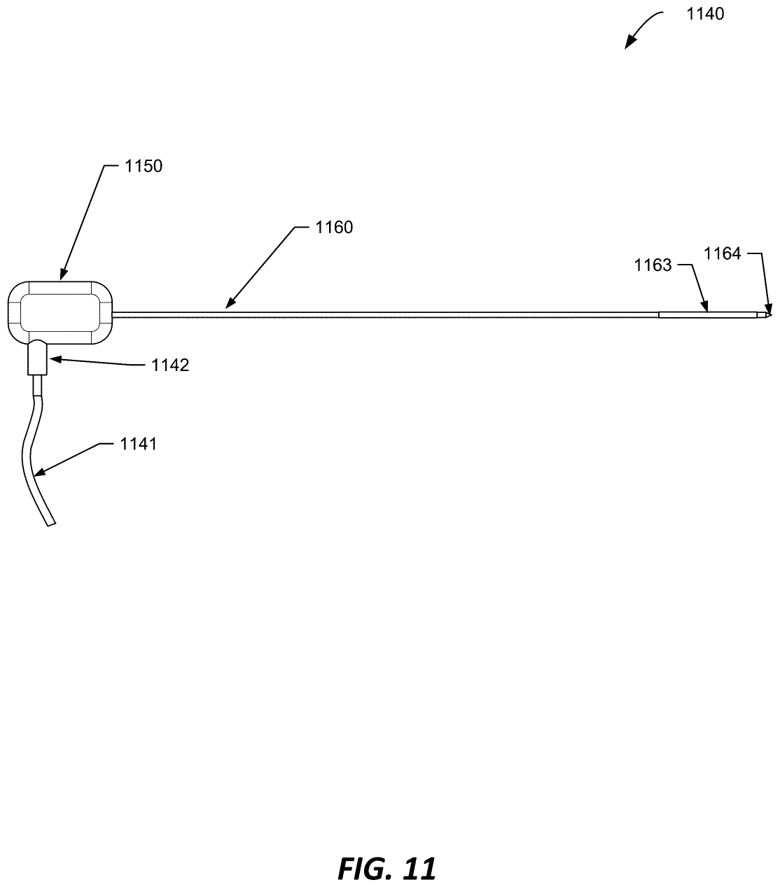

FIG. 10 depicts a block diagram of components of a needle ablation monitoring system 1000, according to various implementations. Needle ablation monitoring system 1000 includes a needle ablation monitoring device 1040. The needle ablation monitoring device 1040 includes an ablation probe 1060 (also sometimes referred to herein as an “ablation needle”) having a radiating portion 1063 with a tip 1064. An example of a suitable ablation needle is the ablation probe made by Covidien Emprint. The radiating portion 1063 is the portion of the ablation needle or probe 1060 from which a substantial portion of the modulated microwave signals is emitted. The length of the radiating portion 1063 may be in a range of 1 and 5 cm. In one example, the length of the radiating portion 1063 is about 3 cm. The illustrated example in FIG. 10 is shown at an instant in time when needle tip 1061 is inserted into a subject 101. It would be understood that subject 101 may not be present at other times such as when the system is not in operation. Needle ablation monitoring device 1140 shown in FIG. 11 is an example of an implementation of the needle ablation monitoring device 1040 shown in FIG. 10.

Needle ablation monitoring system 1000 also includes an ultrasonic detector array assembly 1010 having an ultrasonic detector array 1012 with a plurality of ultrasonic receivers 1013 and a plurality of integral pre-amplifiers 1018 in electronic communication (e.g., one-to-one correspondence) with the ultrasonic receivers 1013 to receive parallel analog acoustic signals. Needle ablation monitoring system 1000 also includes one or more data acquisition devices (DAQs) 1030 with digitizers in electronic communication (e.g., one-to-one correspondence) with the ultrasonic receivers 1013 to receive parallel analog acoustic signals.

Needle ablation monitoring system 1000 also includes an arbitrary waveform generator (AWG) 1020 for generating a chip signal sweeping over a frequency range and duration, a microwave source 1024 for providing a continuous wave (CW) microwave signal, and a mixer 1022 connected to the AWG 420 and the microwave source 1024 to mix the microwave carrier signal (e.g., 2.4 GHz carrier signal) from the microwave source 1024 with the chirp signal (e.g., 150 μs linear chirp signal from 0.15-1.0 MHZ) from the AWG 1020 to generate modulated microwave signals. Needle ablation monitoring system 1000 also optionally includes a power amplifier 1026 connected to the mixer 1022 to provide a higher-power modulated microwave signal. In some cases, the modulated microwave signal may be amplified to the level of between 100 and 300 W. In one example, the modulated signal is amplified to about 200 W. Some examples of microwave carrier signals that may be used include a 2.4 GHz carrier signal, 900 MHz carrier signal, 9 GHz carrier signal, and a tunable carrier signal in the range of 1-10 GHz. Some examples of chirp signals that may be used include a 150 μs linear chirp signal from 0.15-1.0 MHz, 0.05-1.0 MHz, 0.05-2.0 MHz, and a quadratic chirp signal in the range of 0.05-2.0 MHz. Some examples of durations of the chirp signal include 50 μs, 200 μs, and 400 μs. Some examples of amplification that may be implemented include 50 W, 100 W, 200 W, and 400 W. According to one aspect, amplification may be tuned during the ablation procedure to, e.g., control the rate of ablation.

According to one aspect, the microwave source may be adjustable to provide a tunable signal. The microwave source may be controlled to adjust the tunable carrier signal during the ablation procedure to control the size of the heated region. For example, lower frequencies are able to penetrate deeper into tissue. By tuning the frequency during the procedure, the size of the heated region could be controlled to optimally treat a desired region. Needle ablation monitoring system 1000 also includes a computing device 1090 and one or more DAQs 1030 connected to the computing device 1090 to receive control signals and/or send digital acoustic signal data. The DAQ(s) 1030 are also connected to the AWG 1020 to send signals to trigger chirp signals. For the sake of brevity, the prior discussion of the ultrasonic detector array assembly 110, ultrasonic detector array 112, ultrasonic receivers 113, pre-amplifiers 118, data acquisition devices (DAQs) 130, arbitrary waveform generator 120, power amplifier 126, and computing device 190 with regards to FIG. 1 may be assumed to be equally applicable, unless indicated otherwise, to similar or analogous counterparts of those elements in FIG. 10 that share the same last two digits in their respective callouts as in FIG. 1.

The PA 1026 is coupled to the ablation needle 1071 either directly or via a separate coupler (e.g., a coaxial connector such as a SMA). During operation, the modulated microwave signals at acoustic frequencies propagate to the tip 1073 until they are scattered at the tip 1073. The emissions from the tip 1073 generate heating in the subject 101 near the tip 1073 of the inserted ablation needle 1071 to generate thermoacoustic signals from thermoelastic expansion. For example, the subject may be heated within a region of 50-150° C. around the tip 1073. The thermoacoustic signals are coupled through an acoustic medium to the ultrasonic receiver elements in the ultrasonic detector array 1012. The parallel analog thermoacoustic signals are digitized and recorded by the integrated digitizers and the thermoacoustic data is transmitted to computing device 1090. Tissue heating is proportional to the square of the applied electric field, so the second-harmonic thermoacoustic signals are at twice the modulation frequency. The thermoacoustic signals recorded can be cross-correlated with the second-harmonic chirp to recover an effective pulse response.

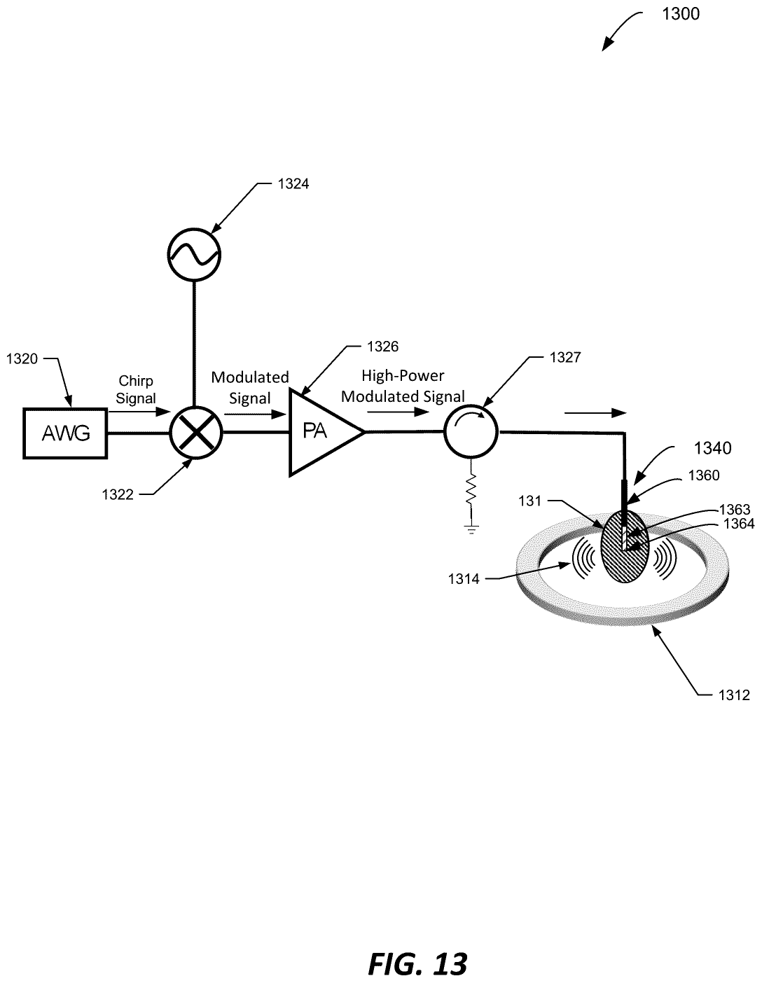

In one embodiment, system 1000 also includes a microwave circulator (e.g., microwave circulator 1327 in FIG. 13) between the power amplifier 1026 and the ablation needle 1071. The microwave circulator is used to direct the reflected microwave signals from the ablation needle 1071 back to a microwave load rather than back to the power amplifier 1071. This ensures the power amplifier 1026 is not damaged from strong signals reflecting into the output port.

During imaging operations, the ultrasonic detector array 1012 is acoustically coupled with the subject 101. For example, the needle ablation monitoring system 1000 may include a tank of acoustic coupling medium and the subject 101 may be at least partially submerged in the medium during image acquisition. In another implementation, one or more packages or containers of acoustic coupling medium (e.g., water packs) may be placed in contact with the subject 101 and the ultrasonic receivers 1013. In addition, needle ablation monitoring system 1000 may include one or more components (e.g., a stepper motor) coupled to the ultrasonic detector array 1012 to enable its movement along a z-axis to one or more elevational positions.

The computing device 1090 includes one or more processors and/or other circuitry, an optional display connected to the processor(s), and a computer readable media (CRM) connected to the processor(s) or other circuitry. Computing device 1090 is connected to DAQ(s) 1030 to receive ultrasound data. Processor(s) and/or other circuitry are connected to CRM to store and/or retrieve data. The one or more processor(s) and/or other circuitry are connected to optional display for, e.g., displaying one or more images. Although not shown, computing device 1090 may also include a user input device for receiving data from an operator of needle ablation monitoring system 1000. The computing device 1090 may be, for example, a personal computer, an embedded computer, a single board computer (e.g., Raspberry Pi or similar), a portable computation device (e.g., tablet), or any other computation device or system of devices capable of performing the functions described herein. Optionally, computing device 1090 may be in communication with a controller to send control signals with control and synchronization data for synchronizing the functions of the system components.

In one aspect, the one or more processors of computing device 1090 and/or other circuitry may execute instructions stored on the CRM to perform one or more imaging operations such as a biopsy needle localization operation. For example, processor(s) and/or other circuitry may execute instructions for: 1) communicating control signals to one or more components of needle ablation monitoring system 1000 to generate the modulated microwave signal and record thermoacoustic signals, 2) reconstructing one or more thermoacoustic images of a heating profile in tissue around the needle tip, and 3) determine one or more adjusted ablation parameters.

The electrical connections between components of needle ablation monitoring system 1000 may be in wired and/or wireless form. One or more of the electrical connections between components of needle ablation monitoring system 1000 may be able to provide power in addition to communicate signals. During operation, digitized data may be first stored in an onboard buffer, and then transferred to the computing device 1090 of needle ablation monitoring system 1000, e.g., through a universal serial bus 2.0.

During an exemplary needle ablation monitoring procedure, subject 101 is placed in contact with an acoustic medium (e.g., submerged in a water immersion tank) and the AWG 420 is activated to generate a chirp signal to modulate the microwave source 1024 to generate a modulated microwave signal. The mixer 1022 mixes the microwave carrier signal with a chirp signal from the AWG 1020 to generate modulated microwave signals. The modulated microwave signals are amplified by the PA 1026. The amplified modulated microwave signals propagate along the ablation needle 1071 are scattered at the tip 1073. The emissions heat an area of the subject near the tip 1073 which generates thermoacoustic signals from thermoelastic expansion. The ultrasonic detector array 1012 detects the thermoacoustic signals. The parallel analog thermoacoustic signals are digitized and recorded by the integrated DAQ digitizers and the thermoacoustic data is transmitted to computing device 1090. The computing device 1090 may determine one or more ablation parameters from the thermoacoustic data. In some cases, the ablation parameters may be used to adjust the ablation process settings such as duration and microwave power.

### Examples of Biopsy Needle Localization Apparatus

In various embodiments, a needle ablation monitoring apparatus includes a microwave ablation probe/needle that emits modulated microwave signals. Transient heating in tissue generates thermoacoustic signals by thermal expansion. The thermoacoustic signals are used to generate one or more images of the local heating profile around the ablation needle. Although examples are described with respect to microwave radiation, other implementations could use other types of radiation such as other radio frequency radiation, optical waves, or ultrasonic waves.

FIG. 11 depicts a side view of a needle ablation monitoring device 1140 for use with thermoacoustic tomography (TAT) ablation monitoring, according to embodiments. Needle ablation monitoring device 1140 includes a body 1150 and an ablation needle 1160 with a proximal end within the body 1150 and a distal end including a radiating portion 1163 from which modulated microwaves at acoustic frequencies are emitted during operation. The radiating portion 1163 includes a tip 1164 at a distal end that can be inserted into a subject (e.g., subject 101 in FIG. 10) being imaged. The ablation needle 1160 may be a solid needle. In one example, the ablation needle may be made of various types of materials such as, e.g., carbon fiber, stainless steel, ceramic or plastic. The body 1150 is formed of a plastic material and is of a generally rectangular shape. Other shapes and materials may be used in other implementations. Needle ablation monitoring device 1140 also includes a cable 1141 (e.g., a coaxial cable) with an electrical connector 1142 at a proximal end that is connected to the proximal end of the ablation needle 1160 within the body 1150. The distal end of cable 1141 is connected to a power amplifier (PA) (e.g., PA 1026 in FIG. 10) or with a mixer (e.g., mixer 1022 in FIG. 10).

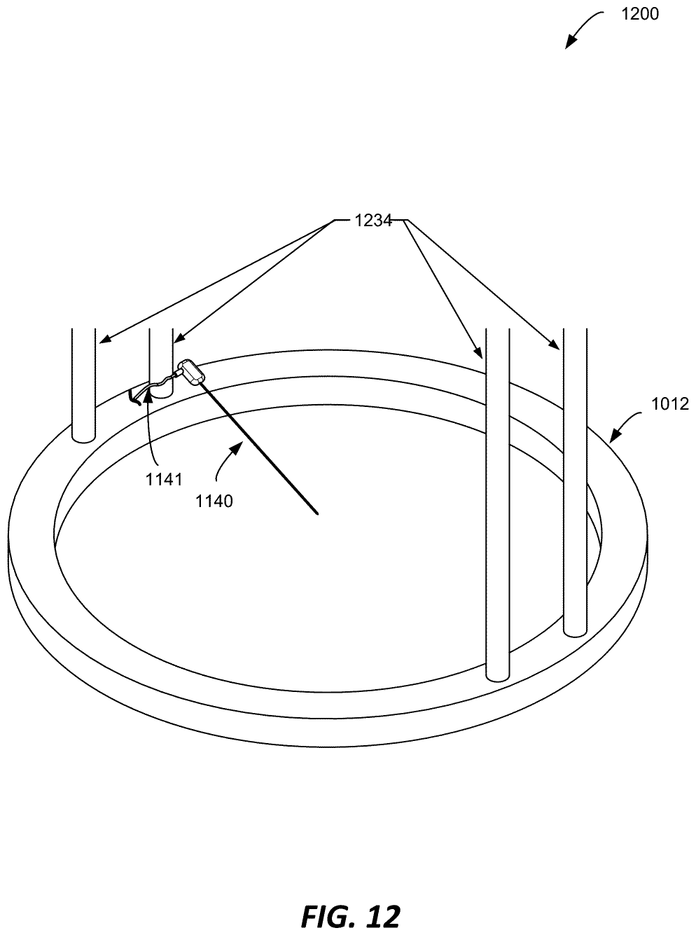

In some embodiments, a needle ablation monitoring system includes the components of the needle ablation monitoring system 1000 shown in FIG. 10 where the needle ablation monitoring device 1040 is the example implementation of the needle ablation monitoring device 1140 shown in FIG. 11. FIG. 12 depicts an example of such an embodiment.

FIG. 12 depicts an isometric view of components of a needle ablation monitoring system 1200, according to embodiments. Needle ablation monitoring system 1200 includes the components of the needle ablation monitoring system 1000 shown in FIG. 10 with the example implementation of the needle ablation monitoring device 1140 shown in FIG. 11. Needle ablation monitoring system 1200 also includes a plurality of conduits 1234 within which wiring (e.g., one or more cables) passes between ultrasonic detector array 1012 and the DAQs 1030 and/or other system components. The distal end of the cable 1141 is connected to the PA 1026 and/or the mixer 1022.

FIG. 13 depicts a block diagram of components of a needle ablation monitoring system 1300, according to various implementations. FIG. 13 shows the flow for microwave ablation monitoring using an ablation probe 1360 and parallel thermoacoustic signal detection. Needle ablation monitoring system 1300 includes a needle ablation monitoring device 1340 with an ablation needle or probe 1360 having a radiating portion 1363 with a tip 1364 at distal end. The illustrated example in FIG. 13 is shown at an instant in time when needle tip 1361 is inserted into a subject 131. It would be understood that subject 131 may not be present at other times such as when the system is not in operation. For the sake of brevity, the prior discussion of the components of needle ablation monitoring system 1000 with regards to FIG. 10 may be equally applicable, unless indicated otherwise, to similar or analogous counterparts of those elements in FIG. 13 that share the same last two digits in their respective callouts as in FIG. 10.

Needle ablation monitoring system 1300 also includes an ultrasonic detector array 1312 with a plurality of ultrasonic receivers and a plurality of integral pre-amplifiers connected (e.g., one-to-one correspondence) to the ultrasonic receivers to receive parallel analog acoustic signals. Needle ablation monitoring system 1300 also includes an arbitrary waveform generator (AWG) 1320 for generating a chip signal sweeping over a frequency range and duration, and a microwave source 1324 for providing a continuous wave (CW) microwave signal. Needle ablation monitoring system 1300 also includes a mixer 1022 connected to the AWG 1320 and the microwave source 1324 to mix the microwave carrier signal (e.g., 2.4 GHz carrier signal) from the microwave source 1324 with the chirp signal (e.g., 150 μs linear chirp signal from 0.15-1.0 MHZ) from the AWG 1320 to generate modulated microwave signals. Needle ablation monitoring system 1300 also includes a power amplifier 1026 connected to the mixer 1322 to provide a higher-power modulated microwave signal. The power amplifier 1326 may amplify the signal to a 200 W modulated signal, for example. Needle ablation monitoring system 1300 may include one or more components (e.g., a stepper motor) coupled to the ultrasonic detector array 1312 to enable its movement along a z-axis to one or more elevational positions. Needle ablation monitoring system 1300 may also include one or more DAQs (e.g., DAQs 1030 in FIG. 10) with digitizers connected (e.g., one-to-one correspondence) to the ultrasonic receivers to receive parallel analog acoustic signals. Needle ablation monitoring system 1300 also includes a microwave circulator 1327 between the power amplifier 1026 and the ablation needle 1071. Microwave circulator 1327 is used to direct the reflected microwave signals from the ablation needle 1371 back to a microwave load rather than back to the power amplifier 1371. This can avoid damage to the power amplifier 1026 from strong signals reflecting into the output port.

During an exemplary needle ablation monitoring procedure, subject 131 is placed in contact with an acoustic medium (e.g., submerged in a water immersion tank) and the microwave source 1324 is activated to generate a continuous microwave signal. Mixer 1322 mixes the microwave carrier signal with a chirp signal from the AWG 1320 to modulate the microwave source 1324 to generate modulated microwave signals. The modulated microwave signals arc amplified by the PA 1326. The microwave circular 1327 directs the reflected microwave signals from the ablation needle 1371 back to a microwave load. The amplified modulated microwave signals propagate along the ablation needle 1371 are scattered at the tip 1373. The emissions heat an area of the subject near the tip 1373 which generates thermoacoustic signals from thermoelastic expansion. The ultrasonic detector array 1312 detects the thermoacoustic signals. The parallel analog thermoacoustic signals may be digitized and recorded by the integrated DAQ digitizers and the thermoacoustic data is transmitted to a computing device (e.g., computing device 1090 in FIG. 10). The computing device may determine one or more ablation parameters from the thermoacoustic data. In some cases, the ablation parameters may be used to adjust the ablation process settings such as duration and microwave power level.

### Experimental Data

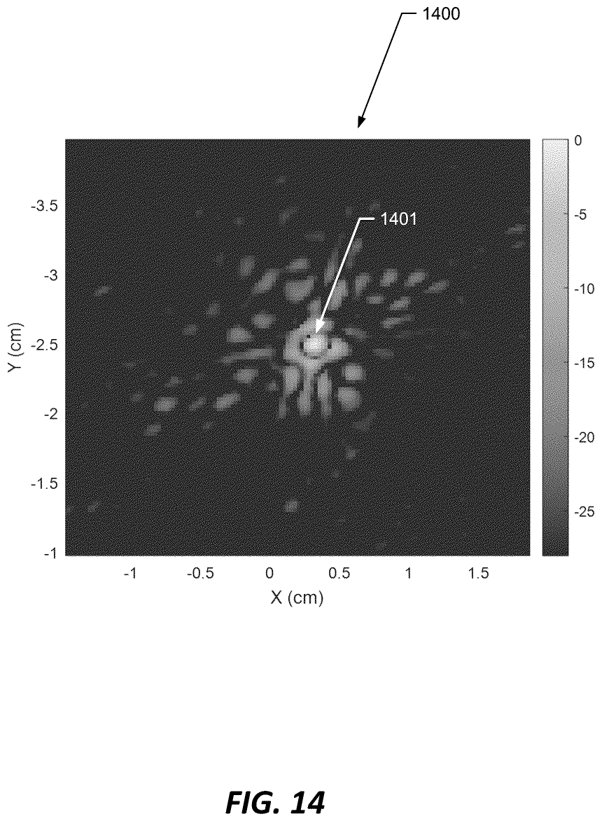

FIGS. 14-17 illustrate experimental results from a needle ablation monitoring procedure performed by needle ablation monitoring system 1300 in FIG. 13. In the needle ablation monitoring procedure, a 2.4 GHz carrier signal from the microwave source 1324 was mixed with a 150 μs linear chirp signal from 0.15-1.0 MHz from the AWG 1320 using the mixer 1322. The modulated signal was then amplified to 200 W using the PA 1326 and the modulated signal was coupled to the ablation needle/probe 1371 (e.g., commercial ablation probe made by Covidien Emprint). Tissue heating is proportional to the square of the applied electric field, so the resulting second-harmonic thermoacoustic signals were at twice the modulation frequency (from 0.3-2.0 MHz). These thermoacoustic signals are detected using the ultrasonic detector array 1312 (e.g., a 512-element array), and then cross-correlated with the second-harmonic chirp to recover an effective pulse response. In one implementation, each image may be acquired in about 20 seconds. Since conventional needle ablation procedures require several minutes, the ablation monitoring approach of this implementation allowed for multiple images to be obtained in the same amount of time that a single image would be acquired in a conventional procedure.

The needle ablation monitoring procedure was used on bovine liver tissue. FIG. 14 is an example of a thermoacoustic image 1400 of bovine liver tissue acquired by needle ablation monitoring system 1300 in FIG. 13. The thermoacoustic image 1400 shows stronger absorption (brighter) near the ablation probe 1401. The needle ablation monitoring system 1300 was used to image the tissue (acquiring a plurality of frames) throughout the five minutes duration of the ablation. The maximum amplitudes of the thermoacoustic images in an area near the ablation probe were determined to evaluate the change in maximum amplitude over time. The amount of reduction in the maximum amplitude is an indicator of the progress of the ablation.

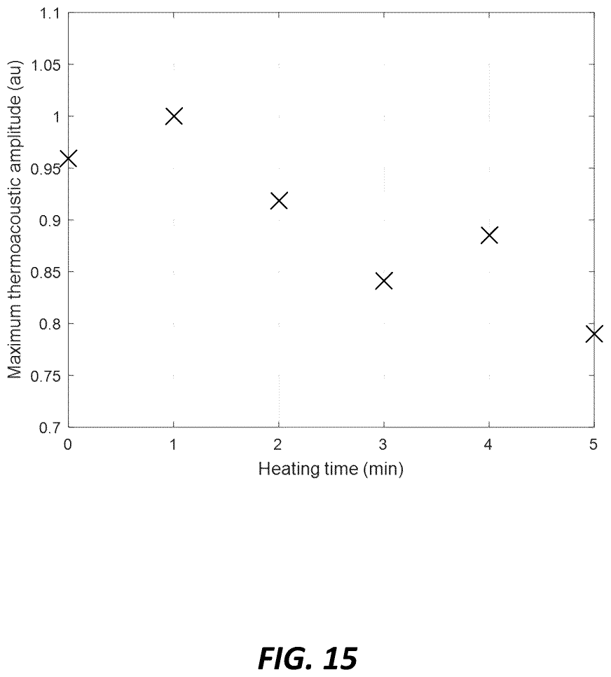

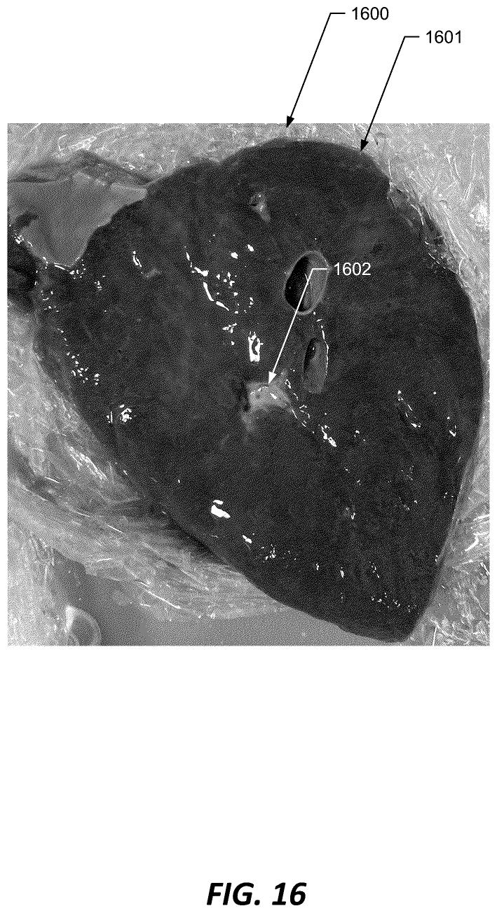

FIG. 15 is a graph of maximum amplitudes of the thermoacoustic images taken every minute of heating time (minutes) over a duration of 5 minutes. The data shows a gradual decrease of about 20% in the maximum amplitude near the ablation probe. This indicates a decrease in tissue conductivity during ablation due to reduction in local water content. In certain embodiments, the needle ablation monitoring procedure may be combined with whole-body UST and TAT imaging to enable imaging and treatment of a variety of conditions. In some cases, this procedure may be used to help ensure ablation is in the target region such as in liver or kidney tumors. FIG. 16 is a photograph 1600 of the bovine liver 1601 after needle ablation showing a treated region 1602.

### Needle Ablation Monitoring Methods

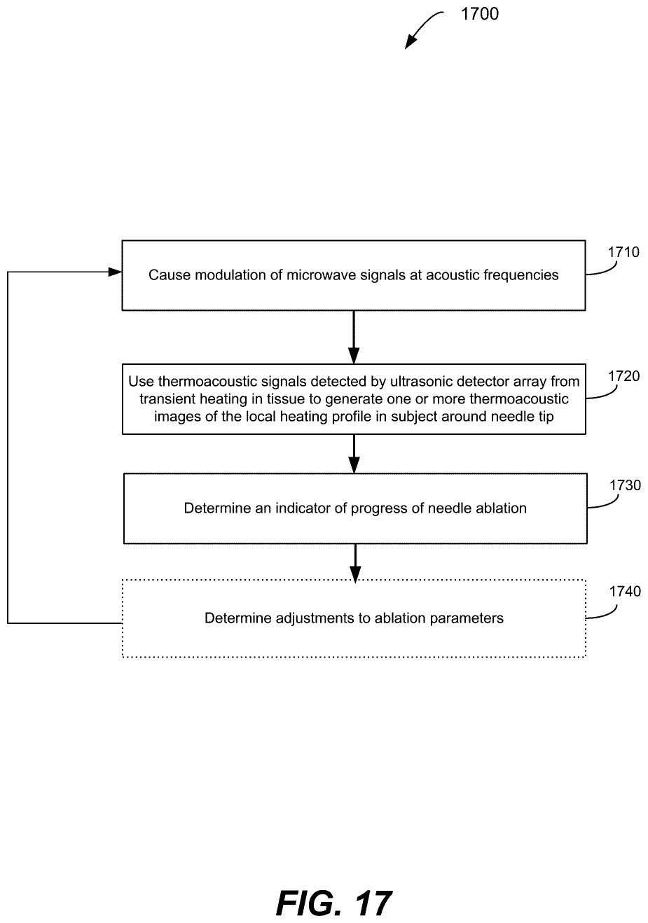

FIG. 17 depicts a flowchart 1700 of a needle ablation monitoring method, according to various embodiments. The needle ablation monitoring method may be implemented by a needle ablation monitoring system such as, for example, the needle ablation monitoring system 1000 in FIG. 10 or the needle ablation monitoring system 1300 in FIG. 13.

At operation 1710, control signals are sent to cause the modulation of microwave signals at acoustic frequencies. Trigger signals are sent to an arbitrary waveform generator (e.g., AWG 1020 in FIG. 10) to emit a chirp signal (e.g., 150 μs linear chirp signal from 0.15-1.0 MHz) and to the microwave source (e.g., microwave source 1024 in FIG. 10) to provide a continuous wave signal (e.g., 2.4 GHz carrier signal). A mixer mixes the microwave carrier signal with the chirp signal to generate a modulated microwave signal and a power amplifier amplifies the signal. The modulate microwave signal is emitted from the ablation probe into the subject and thermoacoustic signals are generated in the heated portion of the subject due to thermoelastic expansion. The ultrasonic detector array detects the thermoacoustic signals. Control signals may be sent automatically by a controller or computing device or may be initiated by manual input from an operator (e.g., turning on a device).

At operation 1720, a plurality of thermoacoustic images over time are reconstructed from the raw thermoacoustic signals detected by the ultrasonic detector array over time and recorded by the one or more DAQs. Image reconstruction may include (i) reconstructing two-dimensional images and/or (ii) reconstructing volumetric three-dimensional images. Image reconstruction includes, at least in part, implementing an inverse reconstruction algorithm. Some examples of inverse reconstruction methods that can be used include: (i) forward-model-based iterative methods, (ii) time-reversal methods, and (iii) back projection methods. A 3D back projection method can be used to reconstruct a 3D volumetric image and a 2D back projection method can be used to reconstruct a 2D image. An example of a back projection method is the universal back-projection process described in U.S. patent application Ser. No. 17/090,752, titled “SPATIOTEMPORAL ANTIALIASING IN PHOTOACOUSTIC COMPUTED TOMOGRAPHY” and filed on Nov. 5, 2020, which is hereby incorporated by reference for this description. Another example of a back-projection method can be found in Anastasio, M. A. et al., “Half-time image reconstruction in thermoacoustic tomography,” IEEE Trans., Med. Imaging 24, pp 199-210 (2005), which is hereby incorporated by reference. In another aspect, a dual-speed-of sound (dual-SOS) photoacoustic reconstruction process may be used. An example of a single-impulse panoramic photoacoustic computed tomography system that employs a dual-SOS photoacoustic reconstruction process is described in U.S. patent application 2019/0307334, titled “SINGLE-IMPULSE PANORAMIC PHOTOACOUSTIC COMPUTED TOMOGRAPHY” and filed on May 29, 2019, which is hereby incorporated by reference. The detected signals may be cross-correlated with the chirp prior to reconstruction to recover a pulse-like representation.

At operation 1730, progress of the needle ablation is determined from the plurality of images. For example, the maximum amplitude of each image near the ablation probe may be determined. The level of decrease in the maximum amplitude over time is a measurement of progress of the ablation. This indicates a decrease in tissue conductivity during ablation due to reduction in local water content.

At optional (denoted by dashed line) operation 1740, adjustments to the ablation parameters (e.g. microwave power and duration) may be determined based on the progress of the needle ablation. For example, if the decrease in maximum amplitude is less than 5% over 5 minutes, the power may be increased. As another example, if the decrease in maximum amplitude is at 75%, the procedure may end. The adjustments are provided as feedback to the ablation process to adjust the microwave signals being emitted at operation 1710.

### IV. Tomographic System

FIG. 18 depicts a block diagram of components of tomography imaging system 1800 for needle localization and/or needle ablation monitoring, according to various embodiments. Tomography imaging system 1800 can perform ultrasound tomography (UST) and/or thermoacoustic tomography (TAT) to generate tomographic images of a subject 21.

Tomography imaging system 1800 includes an arbitrary waveform generator (AWG) 1820 for generating a chirp signal, an energy source 1822 connected to the AWG 1820 that generates energy waves, and a needle 1872 such as a biopsy needle or an ablation probe for inserting in a subject 21 present during imaging procedures. Tomography imaging system 1800 also includes an ultrasonic detector array assembly 1810 with an ultrasonic detector array 1812 that includes a plurality of ultrasonic receivers 1813 that detect ultrasound or thermoacoustic signals and a plurality of pre-amplifiers 1818 connected to the ultrasonic receivers 1813. Tomography imaging system 1800 also includes one or more data acquisition modules (DAQs) 1830 connected to the plurality of pre-amplifiers 1818 and with a plurality of digitizers of the DAQ(s) 1830. Tomography imaging system 1800 also includes a computing device 1890 connected to DAQ(s) 1830. For the sake of brevity, the prior discussion of the components of UST system 100 with regards to FIG. 1 may be assumed to be equally applicable, unless indicated otherwise, to similar or analogous counterparts of those elements in FIG. 18 that share the same last two digits in their respective callouts as in FIG. 1.

Computing device 1890 includes one or more processors and/or other circuitry 1894, an optional (denoted by dashed line) display 1892 connected to the processor(s) 1894, and a computer readable media (CRM) 1896 (e.g., a non-transitory computer readable media) connected to the processor(s) or other circuitry 1894. Computing device 1890 is connected to DAQ(s) 1830 to receive ultrasonic or thermoacoustic data. Processor(s) and/or other circuitry 1894 are connected to CRM 1886 to store and/or retrieve data. The one or more processor(s) and/or other circuitry 1894 are connected to optional display 1892 for, e.g., displaying one or more images. Although not shown, computing device 1890 may also include a user input device for receiving data from an operator of tomography imaging system 1800. The computing device 1890 may be, for example, a personal computer, an embedded computer, a single board computer (e.g., Raspberry Pi or similar), a portable computation device (e.g., tablet), or any other computation device or system of devices capable of performing the functions described herein. Optionally, computing device 1890 may be connected to a controller to send control signals with control and synchronization data.

In one aspect, one or more processors and/or other circuitry 1894 may execute instructions stored on the CRM 1886 to perform one or more operations of a UST or TAT method with needle localization and/or ablation monitoring. For example, processor(s) and/or other circuitry 1894 may execute instructions for: 1) communicating control signals to one or more components of tomography imaging system 1800, 2) reconstructing one or more cross-sectional images of the subject or portions thereof, 3) determining the location of the needle from ultrasonic signals, and 4) determining the progress of needle ablation from ultrasonic signals or from thermoacoustic signals. In one implementation, signals detected by the ultrasonic sensor array 1812 may be streamed to computing device 1890 by DAQ(s) 1830. After which, computing device 1890 may reconstruct a sequence of ultrasound images.

### IV. Example of Components of Computing Device

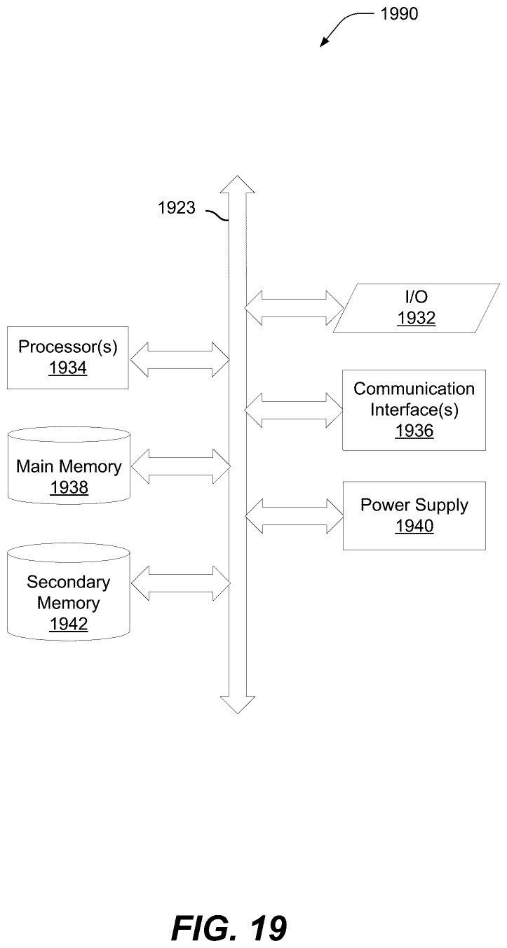

FIG. 19 depicts an example of components of a computing device 1990, according to embodiments. In various examples, computing device 1990 is connected to a tomographic imaging apparatus (e.g., tomographic imaging system 1800 in FIG. 18, UST system 100 in FIG. 1, etc.) to receive signals with tomographic data. In some cases, the computing device 1990 may also send control signals to the tomographic imaging device to control functions. Connections between components of computing device 1990 may be in wireless and/or wired form.

In FIG. 19, computing device 1990 includes a bus 1923 coupled to an input/output (I/O) subsystem 1932, one or more processors 1934, one or more communication interfaces 1936, a main memory 1938, a secondary memory 1942, and a power supply 1940. One of more of these components may be in separate housing. I/O subsystem 1932, includes, or is in connected to, one or more components, which may implement an interface for interacting with human users and/or other computer devices depending upon the application. Certain embodiments disclosed herein may be implemented in program code on computing device 1990 with I/O subsystem 1932 used to receive input program statements and/or data from a human user (e.g., via a graphical user interface (GUI), a keyboard, touchpad, etc.) and to display them back to the user, for example, on a display. The I/O subsystem 1932 may include, e.g., a keyboard, mouse, graphical user interface, touchscreen, or other interfaces for input, and, e.g., an LED or other flat screen display, or other interfaces for output. Other elements of embodiments may be implemented with a computer device like that of computing device 1990 without I/O subsystem 1932. According to various embodiments, the one or more processors 1934 may include a CPU, GPU or computer, analog and/or digital input/output connections, controller boards, etc.

Program code may be stored in non-transitory computer readable media such as secondary memory 1942 or main memory 1938 or both. The one or more processors 1934 may read program code from one or more non-transitory media and execute the code to enable computing device 1990 to accomplish the methods performed by various embodiments described herein, such as a needle localization or needle ablation monitoring method. Those skilled in the art will understand that the one or more processors 1934 may accept source code and interpret or compile the source code into machine code that is understandable at the hardware gate level of the one or more processors 1934.

Communication interfaces 1936 may include any suitable components or circuitry used for communication using any suitable communication network (e.g., the Internet, an intranet, a wide-area network (WAN), a local-area network (LAN), a wireless network, a virtual private network (VPN), and/or any other suitable type of communication network). For example, communication interfaces 1936 can include network interface card circuitry, wireless communication circuitry, etc.

In certain embodiments, computing device 1990 may be part of or connected to a controller that is employed to control functions of a tomographic imaging apparatus such as controlling data acquisition by DAQs. The controller will typically include one or more memory devices and one or more processors. The processor may include a CPU or computer, analog and/or digital input/output connections, stepper motor controller boards, etc.

Certain embodiments are directed to an acoustic and/or thermoacoustic imaging method, system, or devices using the subject matter and techniques described herein. In one aspect, the acoustic signals are coupled into a biopsy needle and detected from outside the body. In another aspect, the resulting needle localization is superimposed on one or more structural images of the body obtained using whole-body ultrasound imaging. In another aspect, a microwave ablation probe is excited using modulated microwave signals, resulting in thermoacoustic signals generated around the needle tip. In another aspect, the generated thermoacoustic signals are detected from outside the body and used to generate images representative of heating profiles around the ablation needle tip.

Modifications, additions, or omissions may be made to any of the above-described embodiments without departing from the scope of the disclosure. Any of the embodiments described above may include more, fewer, or other features without departing from the scope of the disclosure. Additionally, the steps of described features may be performed in any suitable order without departing from the scope of the disclosure. Also, one or more features from any embodiment may be combined with one or more features of any other embodiment without departing from the scope of the disclosure. The components of any embodiment may be integrated or separated according to particular needs without departing from the scope of the disclosure.

It should be understood that certain aspects described above can be implemented in the form of logic using computer software in a modular or integrated manner. Based on the disclosure and teachings provided herein, a person of ordinary skill in the art will know and appreciate other ways and/or methods to implement the present invention using hardware and a combination of hardware and software.

Any of the software components or functions described in this application, may be implemented as software code using any suitable computer language and/or computational software such as, for example, Java, C, C #, C++ or Python, Matlab, or other suitable language/computational software, including low level code, including code written for field programmable gate arrays, for example in VHDL; embedded artificial intelligence computing platform, for example in Jetson. The code may include software libraries for functions like data acquisition and control, motion control, image acquisition and display, etc. Some or all of the code may also run on a personal computer, single board computer, embedded controller, microcontroller, digital signal processor, field programmable gate array and/or any combination thereof or any similar computation device and/or logic device(s). The software code may be stored as a series of instructions, or commands on a CRM such as a random-access memory (RAM), a read only memory (ROM), a magnetic media such as a hard-drive or a floppy disk, or an optical media such as a CD-ROM, or solid stage storage such as a solid state hard drive or removable flash memory device or any suitable storage device. Any such CRM may reside on or within a single computational apparatus, and may be present on or within different computational apparatuses within a system or network. Although the foregoing disclosed embodiments have been described in some detail to facilitate understanding, the described embodiments are to be considered illustrative and not limiting. It will be apparent to one of ordinary skill in the art that certain changes and modifications can be practiced within the scope of the appended claims.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

What is claimed is:

1. An imaging apparatus, comprising:
one or more energy sources configured to generate energy waves;
an arbitrary waveform generator configured to generate one or more chirp signals, the arbitrary waveform generator connected to the one or more energy sources, wherein the one or more chirp signals modulate the energy waves to generate modulated energy waves;
a needle comprising a needle tip, the needle connected to a first energy source of the energy sources, the needle configured to emit the modulated energy waves at or near the needle tip; and
an ultrasonic detector array configured to detect ultrasound signals or thermoacoustic signals based on the modulated energy waves emitted.

2. The imaging apparatus of claim 1, further comprising one or more data acquisition systems configured to record and digitize the ultrasound signals or thermoacoustic signals detected by the ultrasonic detector array.

3. The imaging apparatus of claim 1, wherein:
the needle is a biopsy needle;
the first energy source includes a first ultrasonic transmitter acoustically coupled to the biopsy needle; and
the ultrasonic detector array is configured to detect a first set of ultrasound signals while the biopsy needle emits acoustic signals at the needle tip.

4. The imaging apparatus of claim 3, wherein:
the one or more energy sources includes a second ultrasonic transmitter; and
the ultrasonic detector array is configured to detect a second set of ultrasound signals while the second ultrasonic transmitter is rotated around a subject being imaged.

5. The imaging apparatus of claim 4, further comprising:
a motor; and
a gear coupled to the motor and to the second ultrasonic transmitter, wherein the motor is configured to move the second ultrasonic transmitter along a line, an arc, or a circle.

6. The imaging apparatus of claim 5, further comprising a rotational shaft coupled to the motor and gear, wherein the motor is configured to rotate the at least one ultrasonic transmitter.

7. The imaging apparatus of claim 4, wherein the second ultrasonic transmitter is a single element ultrasonic transducer.

8. The imaging apparatus of claim 4, further comprising a computing device configured to:
generate one or more two-dimensional images of the subject based on ultrasonic signals detected by the ultrasonic detector array while the second ultrasonic transmitter is rotated around a subject being imaged; and
determine a location of the needle tip within the subject from ultrasonic signals detected by the ultrasonic detector array while the biopsy needle emits acoustic signals at the needle tip.

9. The imaging apparatus of claim 8, wherein the computing device is further configured to superimpose the location of the needle tip onto at least one of the two-dimensional images.

10. The imaging apparatus of claim 1, wherein the first energy source is a continuous wave source coupled to the needle.

11. The imaging apparatus of claim 1, wherein the first energy source is a microwave source and wherein the arbitrary waveform generator and the microwave source are configured to provide a modulated microwave signal.

12. The imaging apparatus of claim 11, wherein:
the needle is an ablation probe configured to emit the modulated microwave signal at a radiating portion of the ablation probe; and
the ultrasonic detector array is configured to detect thermoacoustic signals from thermoelastic expansion based on the modulated microwave signal emitted from the radiating portion.

13. The imaging apparatus of claim 12, further comprising a computing device configured to track one or more ablation parameters based on the thermoacoustic signals detected by the ultrasonic detector array.

14. The imaging apparatus of claim 13, wherein at least one of the ablation parameters is an indicator of ablation progress.

15. The imaging apparatus of claim 12, further comprising a mixer for mixing a microwave signal from the microwave source with the one or more chirp signals to generate the modulated microwave signal.

16. The imaging apparatus of claim 1, further comprising a power amplifier connected to the energy source, the power amplifier for amplifying the energy waves.

17. The imaging apparatus of claim 1, wherein the energy source is one of a radio frequency source, a light source, or an ultrasonic source.

18. An imaging method, comprising:
causing an ultrasonic transmitter coupled to a biopsy needle to emit acoustic waves from a needle tip inserted into a subject; and
determining a location of the needle tip within the subject using a first set of ultrasonic signals detected by an ultrasonic detector array while the biopsy needle emits the acoustic waves.

19. The imaging method of claim 18, wherein the location of the needle tip is determined during an in vivo imaging procedure.

20. The imaging method of claim 18, further comprising:
causing the ultrasonic transmitter or another ultrasonic transmitter to move around the subject while the ultrasonic detector array detects a second set of ultrasonic signals; and
generating one or more two-dimensional images of the subject using the second set of ultrasonic signals; and
superimposing the location of the needle tip onto at least one of the two-dimensional images of the subject.

21. An imaging method, comprising:
(a) causing modulation of energy waves from an energy source using one or more chirp signals from an arbitrary waveform generator to generate a modulated energy wave signal, the modulated energy wave signal propagated to a radiating portion of an ablation probe;
(b) generating a plurality of two-dimensional thermoacoustic images from thermoacoustic signals detected by an ultrasonic detector array; and
(c) determining an indicator of ablation progress from the two-dimensional thermoacoustic images.

22. The imaging method of claim 21, further comprising
determining a maximum amplitude of each of the two-dimensional thermoacoustic images; and
determining the indicator of ablation progress from the maximum amplitude of the two-dimensional thermoacoustic images.

23. The imaging method of claim 21, further comprising
determining one or more adjusted ablation parameters based on the indicator of ablation progress; and
adjusting (a) based on the one or more adjusted ablation parameters.

24. The imaging method of claim 21, wherein the one or more adjusted ablation parameters include an adjusted duration and power level of the energy waves from the energy source.

25. The imaging method of claim 21, wherein the energy source is a microwave source and the modulated energy wave signal is a modulated microwave energy wave signal.

26. The imaging method of claim 21, wherein the energy source is one of a radio frequency source, a light source, or an ultrasonic source.
